(19) United States
(12) Patent Application Publication
(10) Pub. No.: US 2016/0369314 A1
(43) Pub. Date: Dec. 22, 2016

(54) ENDOGLUCANASE-INDUCED PRODUCTION OF CELLULOSE OLIGOMERS

(71) Applicant: BASF SE, Ludwigshafen (DE)

(72) Inventors: Mari Granstroem, Kerava (FI); Alois Kindler, Gruenstadt (DE); Antje Spiess, Aachen (DE); Stefanie Kluge, Aachen (DE); Benjamin Bonhage, Lohmar (DE)

(21) Appl. No.: 14/902,046

(22) PCT Filed: Jun. 30, 2014
(86) PCT No.: PCT/EP2014/063876

§ 371 Date: Dec. 30, 2015

(30) Foreign Application Priority Data
Jul. 1, 2013 (EP) 13174460.9

(51) Int. Cl. C12P 19/14; C12P 19/04

(52) U.S. Cl. CPC C12P 19/14; C12P 19/04; C12Y 302/01004

(57) ABSTRACT

The invention relates to a process for the production of cellulose oligomers (cellooligomers) using endoglucanases and to the use in various technical fields of the oligomers produced.

The invention relates to a process for the production of cellulose oligomers (cellooligomers) using endoglucanases and to the use in various technical fields of the oligomers produced.

### BACKGROUND OF THE INVENTION

Cellulose is the most abundant polymer on Earth [Pinkert, Marshet al. Chemical Reviews, 109(12):6712-6728, 2009]; it occurs in plant cell walls in the form of lignocellulose [Teeri, T. T., Trends in Biotechnology, 15(5):160-167, 1997]. The primary and secondary cell wall of plants consists to 10 to 90% of lignocellulose fibers [Weiler, E. W., Allgemeine and molekulare Botanik, Vol. 1. Thieme, Stuttgart, 2008]. In plant cell walls, cellulose, together with lignin and hemicellulose, forms cellulose fibrils. Inside a cellulose fibril there is a crystalline core which is composed of 30 to 100 cellulose molecules arranged in parallel to each other. Cellulose is a structural unit of plant cell walls and responsible for stability and tensile strength, but also for flexibility.

In contrast to sugars and starch, cellulose is not a foodstuff with relevance for human nutrition, and, accordingly, is an ethical renewable raw material for materials and the generation of energy.

Little is known on the production of cellooligomers and their potential use. An addition to foodstuffs and detergents would be feasible. Owing to the high number of hydroxyl groups in the cellooligomers, the latter may be derivatized at a variety of points, whereby their properties may be modified in a targeted manner. Numerous studies on the multi-step conversion of cellulose into biofuels have been published in recent years [Hahn-Haegerdal et al. Trends in Biotechnology, 24(12):549-556, 2006, Jorgensen et al. 2007, Waltz 2008] since the search for renewable energies drives research in this field.

The use of solid catalysts in ionic liquids for the hydrolysis of cellulose has been reported in 2008 [Rinaldi et al., Angewandte Chemie-International Edition, 47(42):8047-8050, 2008]. During the depolymerization of cellulose, however, glucose degradation products such as hydroxymethylfurfural, formic acid and other products which might adversely affect the use of the cellooligomers are generated [Rinaldi et al. Chemsuschem, 3(2): 266-276, 2010]. The good solubility of sugars in ionic liquids complicates the extraction of the sugars and the recycling of the ionic liquid [Rinaldi et al., Angewandte Chemie-International Edition, 47(42):8047-8050, 2008]. If the reaction is terminated prematurely, cellooligomers with a DP of approximately 30 can be prepared after a reaction time of 1.5 h in a yield of 90% [Rinaldi et al., Angewandte Chemie-International Edition, 47(42):8047-8050, 2008].

The object of the present invention is therefore to provide a process with the aid of which cellooligomers can be provided, which, however, does not have the disadvantages of known preparation methods.

### SUMMARY OF THE INVENTION

According to the invention there is provided a novel and surprisingly advantageous process procedure for the production of insoluble cellooligomers by means of endoglucanases. The process according to the invention in particular demonstrates advantages in respect of the production of oligomers with a defined chain length and a narrow chain length distribution of the insoluble cellooligomers, low production of soluble sugars and achieving a cellooligomer chain length near the solubility limit in water.

Since cellulose is a water-insoluble, partially crystalline biopolymer and, accordingly, is difficult to enzymatically hydrolyze in aqueous media [Dadi et al., Biotechnology and Bioengineering, 95(5):904-10, 2006], an additional, in particular suitable, pretreatment, for example by means of ionic liquids, may be carried out. In particular, commercially available purified endoglucanases such as, in particular, from A. niger, B. amyloliquefaciens, T. maritima, are employed for hydrolyzing cellulose.

### DESCRIPTION OF THE FIGURES

FIGS. 1a to 1e show the course of DP_{w} and DPₙ over time for the enzymatic hydrolysis of Avicel by means of endoglucanases from a) A. niger, b) B. amyloliquefaciens, c) T. maritima, d) T. longibrachiatum and e) T. emersonii.

FIGS. 2a to 2e show the course over time of DP_{w} and DPₙ for the enzymatic hydrolysis of alpha-cellulose by means of endoglucanases from a) A. niger, b) B. amyloliquefaciens, c) T. maritima, d) T. longibrachiatum and e) T. emersonii.

FIGS. 3a to 3e show the course over time of the degree of polymerization DP_{w} and DPₙ for the enzymatic hydrolysis of Sigmacell by means of endoglucanases from a) A. niger, b) B. amyloliquefaciens, c) T. maritima, d) T. longibrachiatum and e) T. emersonii.

FIGS. 4a to 4c show the course over time of the degree of polymerization DP_{w} and DPₙ for the two-step enzymatic hydrolysis of Avicel by means of endoglucanases from a) A. niger, b) B. amyloliquefaciens and c) T. maritima following intermediate treatment with ionic liquid (IL Restart).

FIGS. 5a to 5c show the course over time of the degree of polymerization DP_{w} and DPₙ for the two-step enzymatic hydrolysis of alpha-cellulose by means of endoglucanases from a) A. niger, b) B. amyloliquefaciens and c) T. maritima following intermediate treatment with ionic liquid (IL Restart).

### DETAILED DESCRIPTION OF THE INVENTION

### 1. General Definitions

The “DP_{N} value” refers to the number-based degree of polymerization, or number-average chain length, of an oligomer or polymer.

The “DP_{W} value” refers to the weight-based degree of polymerization, or the chain length, of an oligomer or polymer.

The “DP_{N} value” and the “DP_{W} value” are determined experimentally according to the invention in particular using gel permeation chromatography (GPC) under the standard conditions described in greater detail in the experimental part (mobile phase, operating temperature, flow rate, column material).

The “chain length distribution” and the “molar mass distribution” refer to the frequency distribution of the chain length and the molar mass, respectively, determined by GPC. The number-average molar mass M_{N} and the weight-average molar mass M_{w} can be used to quantitatively describe the range of the chain length or molar mass distribution.

Polydispersity is defined as the ratio of M_{w} to M_{N} and is 1 or greater than 1. The lower the polydispersity, the narrower the molar mass distribution.

In the context of the invention, “cellulose” is to be understood in the broad sense in the field of the low-lignin and essentially hemicellulose-free cellulosic materials, from pulp to pure alpha-cellulose. Both pure products and also cellulosic substance mixtures may be employed as long as they do not have a substantial negative effect on the enzymatic reaction. The celluloses may be fully uncrystalline or fully crystalline or exhibit a mean degree of crystallinity (Crl %) as determined by the known determination methods (for example x-ray diffraction). The “DP_{N} value” of cellulose employed may be for example in the range of approximately 30 to 150. The “DP_{W} value” may be in the range of, for example, 120 to 500.

A “cellooligomer” is an oligomer composed of a plurality of β-1,4-glycosidically linked glucose units with a DP_{N} value in the range of 10 to 70.

Endoglucanases (E.C.3.2.1.4) are enzymes which cleave randomly within a cellulose molecule. They attack in the amorphous regions of cellulose. Random chain cleavage gives rise to two cellulose chains, in most cases with different lengths. This results in the rapid reduction of the DP_{W} and in the increase in reducing ends [Lynd et al., Microbiology and Molecular Biology Reviews, 66(3):506-577, 2002].

One endoglucanase activity unit is defined as the amount of enzyme required for generating one mmol glucose equivalent of reducing sugars per minute from carboxymethylcellulose at 40° C. and a pH of 4.5 or 6.

Beta-glucosidase (E.C. 3.2.1.21, or beta-1,6-glucosidase) is a glucosidase enzyme which acts on β1->4 bonds between two glucose or substituted glucose molecules. It is an exocellulase with specificity for a multiplicity of beta-D-glycosidic substrates. It catalyzes the hydrolysis of nonreducing terminal residues in beta-D-glucosides with liberation of glucose.

Bacillus amyloliquefaciens is a Gram-positive rod-shaped bacterium which was discovered by J. Fukomoto in 1943 [Fukomoto, J., J. Agric. Chem. SOC. Jpn., 19; 487-503; 1943]. It was characterized by a group of scientists, and declared as a separate species, only in 1987 [Priest et al. International Journal of Systematic Bacteriology, 37(1):69-71, 1987]. B. amyloliquefaciens was isolated from soil and has a size of 0.7 μm to 0.9 μm by 1.8 μm to 3.0 μm. The cells show peritrichous flagellation and are mobile and form chains. The temperature optimum is between 30° C. and 40° C. Growth ceases below 15° C. [Priest et al., International Journal of Systematic Bacteriology, 37(1):69-71, 1987]. The amylases prepared from B. amyloliquefaciens are enzymes which are employed in industry for starch hydrolysis. In the American Type Culture Collection, B. amyloliquefaciens has the number 23350.

Aspergillus niger is a filamentous fungus which grows aerobically [Schuster et al. Applied Microbiology and Biotechnology, 59(4-5):426-435, 2002]. A. niger is found in nature in soil, waste, compost and rotted plant material. A. niger grows at temperatures from 6° C. to 47° C. and in a pH range from 1.4 to 9.8 [Reiss, J., Schimmelpilze Lebensweise, Nutzen, Schaden, Bekaempfung. Springer, 1986]. It produces black shaded conidiospores that disperse in the air. In industry, A. niger is employed predominantly for the production of citric acid and gluconic acid [Roukas, T., Journal of Industrial Microbiology and Biotechnology, 25 (6):298-304, 2000].

Thermotoga maritima is a rod-shaped Gram-negative strictly anaerobic bacterium with a free outer cell membrane. T. maritima was discovered by R. Huber in a volcano off Italy in 1986. The growth optimum of this bacterium is at 80° C. No growth occurs below 55° C. T. maritima has a length of 1.5 μm to 11 μm and a width of 0.6 μm. It shows subpolar monotric flagellation and can therefore move freely [Huber et al., Archives of Microbiology, 144(4):324-333, 1986]. T. maritima grows on simple and complex carbohydrates, such as glucose, sucrose, starch, cellulose and xylan [Nelson, K. E. et al., Nature, 399(6734):323-329, 1999].

Talaromyces emersonii belongs to the family Trichocomaceae, which belongs to the division Ascomycota and was discovered by Stolk in 1965 [Stolk, A. C., et al., Journal of Microbiology and Serology, 31(3):262, 1965]. The current name is Rasamsonia emersonii [Houbraken, Spierenburg, Frisvad. Antonie van Leeuwenhoek, 101(2):403-21, 2012].

Trichoderma longibrachiatum belongs to the family Hypocreaceae. This belongs to the division Ascomycota. T. longibrachiatum was discovered by Rifai in 1969 [Rifai, M. A., Mycological Paters No. 116, Commonwealth Mycological Institute, 1969]. The species has already been the subject of many studies [Bissett, J., Canadian Journal of Botany—Revue Canadienne de Botanique, 62(5):924-931, 1984; J., Canadian Journal of Botany—Revue Canadienne de Botanique, 69(11):2357-2372, 1991; J., Canadian Journal of Botany—Revue Canadienne de Botanique, 69(11):2373-2417, 1991; J., Canadian Journal of Botany—Revue Canadienne de Botanique, 69(11):2418-2420, 1991].

### 2. Special Embodiments of the Invention

The present invention relates in particular to the following embodiments:
1. Process for the production of cellooligomers, where
   a) cellulose or a cellulosic starting material is cleaved hydrolytically using at least one endoglucanase (EG) (E.C.3.2.1.4), in particular a microbial EG, such as, for example, from bacteria or fungi, in an aqueous reaction medium, and
   b) the reaction product, which comprises one or more cellooligomers, i.e. a cellooligomer fraction, is isolated from the reaction medium.
2. Process according to embodiment 1, wherein the cellooligomer(s) formed has (have) a number-average chain length or a number-average degree of polymerization DP_{N} in the range of from 10 to 100.
3. Process according to any of the preceding embodiments, wherein the cellooligomer(s) formed has (have) a DP_{N} value in the range of from 15 to 50, such as, for example, 20 to 45, 25 to 40, or 30 to 35.
   The chain length distribution of cellooligomers produced in accordance with the invention may, for example, be in the range of from 5 to 500, 10 to 4000 15 to 300 20 to 2000 or 25 to 150, without being limited thereto.
4. Process according to any of the preceding embodiments, wherein the EG is a natural or recombinantly produced, optionally genetically modified enzyme from microorganisms of the genus Bacillus, Aspergillus or Thermotoga, in particular the species Bacillus amyloliquefaciens, Aspergillus niger or Thermotoga maritima, or a combination of at least two of these natural or recombinant enzymes.
5. Process according to any of the preceding embodiments, wherein the enzymatic hydrolysis is carried out in an aqueous medium at a pH in the range of approximately 3 to 8, in particular 4 to 7 or 5 to 6 and/or at a temperature in the range of from 20 to 90, in particular from 30 to 80 or from 40 to 70° C. and/or over a duration of 0.1 to 100 hours, in particular 1 to 72 or 1 to 48 hours.
6. Process according to any of the preceding embodiments, wherein at least one EG is applied at a concentration of approximately 0.01 to 100, such as, for example, 1 to 50, 1.5 to 30 or 2 to 10 U/ml reaction mixture.
7. Process according to any of the preceding embodiments, wherein cellulose is employed at a concentration in the range of from 0.1 to 5 or from 1 to 4 or from 2 to 3% (w/v) based on the total volume of the reaction mixture.
8. Process according to any of the preceding embodiments, wherein the cellulose
   a1) is subjected to a pretreatment step by which the crystallinity of the cellulose is reduced, and
   a2) the cellulose of step 1a) is hydrolyzed enzymatically using the EG.
9. Process according to embodiment 8, wherein the crystallinity of the cellulose is reduced by treatment in step a1) using ionic liquid, acid and/or mechanical energy input.
10. Process according to embodiment 9, wherein the ionic liquid is selected among salts which are liquid below a temperature of 100° C. such as, in particular, 1-ethyl-3-methylimidazolium acetate (EMIM Ac) and 3-methyl-N-butylpyridinium chloride ([C4mpy]Cl).
11. Process according to embodiment 10, wherein, in step a1), cellulose is introduced into the ionic liquid, dissolved therein, optionally under thermal action, such as, for example, 20 to 80, 25 to 60 or 30 to 50° C. and subsequently precipitated by adding water, an organic solvent or a mixture thereof, the precipitate is separated off, optionally washed and liquid is optionally removed.
12. Process according to embodiment 9, wherein, in step a1), the acid treatment is carried out with concentrated phosphoric acid.
13. Process according to embodiment 9, wherein, in step a1), the mechanical treatment is carried out using a ball mill, for example with 1 mm glass beads, and/or a wattage in the range of approximately 200 to 600 or 300 to 500 or approximately 400 W.
14. Process according to any of the preceding embodiments, wherein the treatment steps, in particular steps a1) and a2), are repeated once or more than once before the reaction product is isolated.
15. Process according to one of the preceding embodiments, wherein the reaction is additionally carried out in the presence of a beta-glucosidase.
16. Use of a cellooligomer, produced by a process according to any of the preceding embodiments, as additive to foodstuffs and feedstuffs, cosmetics or pharmaceuticals, as a detergent additive, as a rheology modifier and as a starting material for organic syntheses.

### 3. Further Configurations of the Invention

3.1 Enzymes—Endoglucanases which can be Used

The present invention is not limited to the specifically disclosed or used proteins or enzymes with endoglucanase activity, but rather also extends to functional equivalents of these.

Nonlimiting examples of amino acid sequences, of enzymes used in accordance with the invention, which are known from the literature are specified hereinbelow.

Thermotoga maritima: (SEQ ID NO:1)

Nelson K. E. et al., “Evidence for lateral gene transfer between Archaea and bacteria from genome sequence of Thermotoga maritima.” Nature 399:323-329(1999)

Uniprot: Q9X274

```
                10 20 30 40
MNNTIPRWRG FNLLEAFSIK STGNFKEEDF LWMAQWDFNF
                50 60 70 80
VRIPMCHLLW SDRGNPFIIR EDFFEKIDRV IFWGEKYGIH
                90 100 110 120
ICISLHRAPG YSVNKEVEEK TNLWKDETAQ EAFIHHWSFI
              130 140 150 160
ARRYKGISST HLSFNLINEP PFPDPQIMSV EDHNSLIKRT
              170 180 190 200
ITEIRKIDPE RLIIIDGLGY GNIPVDDLTI ENTVQSCRGY
              210 220 230 240
IPFSVTHYKA EWVDSKDFPV PEWPNGWHFG EYWNREKLLE
              250 260 270 280
HYLTWIKLRQ KGIEVFCGEM GAYNKTPHDV VLKWLEDLLE
              290 300 310 320
IFKTLNIGFA LWNFRGPFGI LDSERKDVEY EEWYGHKLDR
KMLELLRKY
```

Aspergillus niger: (SEQ ID NO:2)

van Peij N. N., et al., “The transcriptional activator XlnR regulates both xylanolytic and endoglucanase gene expression in Aspergillus niger.” Appl. Environ. Microbiol. 64:3615-3619(1998)

Uniprot: O74705

```
                10 20 30 40
MKLPVSLAML AATAMGQTMC SQYDSASSPP YSVNQNLWGE
                50 60 70 80
YQGTGSQCVY VDKLSSSGAS WHTEWTWSGG EGTVKSYSNS
                90 100 110 120
GVTFNKKLVS DVSSIPTSVE WKQDNTNVNA DVAYDLFTAA
              130 140 150 160
NVDHATSSGD YELMIWLARY GNIQPIGKQI ATATVGGKSW
              170 180 190 200
EVWYGSTTQA GAEQRTYSFV SESPINSYSG DINAFFSYLT
              210 220 230
QNQGFPASSQ YLINLQFGTE AFTGGPATFT VDNWTASVN
```

Bacillus amyloliquefaciens (SEQ ID NO:3):

Zhang G., et al., “Complete Genome Sequence of Bacillus amyloliquefaciens TA208, a Strain for Industrial Production of Guanosine and Ribavirin.” J. Bacteriol. 193:3142-3143(2011)

Uniprot: F4E3N1

```
                10 20 30 40
MKHTMELIKK LVSIPSPTGN TYEVIAYTES LLKDWGVSSY
                50 60 70 80
RNRKGGLFVT IPGRDDKKHR LLTAHVDTLG AMVKEIKADG
                90 100 110 120
RLKIDLIGGF RYNSIEGEYC EIQTSSGKTY TGTILMHQTS
              130 140 150 160
VHVYKDAGKA ERNQENMEVR LDEHVRTKEE TSDLGIRVGD
              170 180 190 200
FISFDPRVQI TPSGFIKSRH LDDKASVALL LDLIRRITEE
              210 220 230 240
KIDLPYTTHF LISNNEEIGY GGNSNIPPET VEYLAVDMGA
              250 260 270 280
IGDGQSTDEY TVSICVKDAS GPYHYQLRKH LAGLAERYGI
              290 300 310 320
DYQLDIYPYY GSDASAAIRS GHDIVHGLIG PGIDASHAFE
              330 340
RTHELSLLNT AKLLHRYVLS PMA
```

“Functional equivalents” or analogs of the enzymes used specifically in accordance with the invention or described herein are polypeptides which differ from them within the scope of the present invention but which retain the desired biological activity, such as, for example, endoglucanase activity.

Thus, for example, “functional equivalents” are understood as meaning enzymes which, in the endoglucanase activity assay employed, have an activity of an enzyme comprising an amino acid sequence defined herein which is at least 10% or 20%, such as, for example, at least 50% or 75% or 90%, higher or lower. In addition, functional equivalents are preferably stable between pH 2 to 11 and advantageously have a pH optimum in the range of from pH 3 to 10 and a temperature optimum in the range of from 25° C. to 95° C. or 20° C. to 70° C., such as, for example, approximately 45 to 60° C. or approximately 50 to 55° C.

The endoglucanase activity can be detected with the aid of various known assays. Without imposing any limitation, mention may be made of an assay using a reference substrate, such as, for example, carboxymethylcellulose, under standardized conditions at 40° C. and a pH of 4.5 or 6.

In accordance with the invention, “functional equivalents” are, in particular, also understood as meaning “mutants” which have an amino acid other than the specifically mentioned amino acid at at least one sequence position of the abovementioned amino acid sequences while retaining one of the abovementioned biological activities. Thus, “functional equivalents” comprise the mutants obtainable by one or more amino acid additions, substitutions, deletions and/or inversions, it being possible for the abovementioned modifications to occur at any sequence position as long as they result in a mutant with the property profile according to the invention. Functional equivalence exists in particular also when the reactivity patterns between mutant and unmodified polypeptide agree in terms of quality, i.e. when identical substrates are converted at different rates. Examples of suitable amino acid substitutions are compiled in the table which follows:

| Original residue | Substitution examples |
|---|---|
| Ala | Ser |
| Arg | Lys |
| Asn | Gln; His |
| Asp | Glu |
| Cys | Ser |
| Gln | Asn |
| Glu | Asp |
| Gly | Pro |
| His | Asn; Gln |
| Ile | Leu; Val |
| Leu | Ile; Val |
| Lys | Arg; Gln; Glu |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

“Functional equivalents” in the above sense are also “precursors” of the described polypeptides and “functional derivatives” and “salts” of the polypeptides.

In this context, “precursors” are natural or synthetic precursors of the polypeptides with or without the desired biological activity.

The expression “salts” is understood as meaning not only salts of carboxyl groups, but also acid addition salts of amino groups of the protein molecules according to the invention. Salts of carboxyl groups can be prepared in a manner known per se and comprise inorganic salts such as, for example, sodium, calcium, ammonium, iron and zinc salts, and salts with organic bases such as, for example, amines, such as triethanolamine, arginine, lysine, piperidine and the like. Acid addition salts such as, for example, salts with mineral acids, such as hydrochloric acid or sulfuric acid, and salts with organic acids, such as acetic acid or oxalic acid, are likewise subject matter of the invention.

Likewise, it is possible to prepare “functional derivatives” of polypeptides according to the invention at functional amino acid side groups or on their N- or C-terminal end with the aid of known techniques. Such derivatives comprise, for example, aliphatic esters of carboxylic acid groups, amides of carboxylic acid groups, obtainable through reaction with ammonia or with a primary or secondary amine; N-acyl derivatives of free amino groups, prepared by reaction with acyl groups; or O-acyl derivatives of free hydroxyl groups, prepared by reaction with acyl groups.

Of course, “functional equivalents” also comprise polypeptides which are available from other organisms, and naturally occurring variants. For example, it is possible by means of sequence alignment to identify regions of homologous sequence regions, and to determine equivalent enzymes based on the specific requirements of the invention.

“Functional equivalents” likewise comprise fragments, preferably single domains or sequence motifs, of the polypeptides according to the invention which have, for example, the desired biological function.

“Functional equivalents” are additionally fusion proteins which comprise one of the abovementioned polypeptide sequences or functional equivalents derived therefrom and at least one further, heterologous sequence which is functionally different therefrom and is in functional N- or C-terminal linkage (i.e. without significant mutual functional impairment of the parts of the fusion protein). Nonlimiting examples of such heterologous sequences are, for example, signal peptides, histidine anchors or enzymes.

“Functional equivalents” also included according to the invention are homologs of the specifically disclosed proteins. They have at least 60%, preferably at least 75%, in particular at least 85%, such as, for example, 90, 91, 92, 93, 94, 95, 96, 97, 98 or 99%, homology (or identity) to one of the specifically disclosed amino acid sequences, calculated by the algorithm of Pearson and Lipman, Proc. Natl. Acad, Sci. (USA) 85(8), 1988, 2444-2448. A percentage homology or identity of a homologous polypeptide according to the invention means in particular percentage identity of the amino acid residues based on the total length of one of the amino acid sequences specifically described herein.

The percentage identities can also be determined with reference to BLAST alignments, algorithm blastp (protein-protein BLAST) or by using the Clustal settings specified hereinbelow.

In case of possible protein glycosylation, “functional equivalents” according to the invention comprise proteins of the type specified hereinabove in deglycosylated or glycosylated form, and modified forms obtainable by altering the glycosylation pattern.

Homologs of the proteins or polypeptides according to the invention can be generated by mutagenesis, i.e. by point mutation, extension or truncation of the protein.

Homologs of the proteins according to the invention can be identified by screening combinatorial libraries of mutants such as, for example, truncation mutants. For example, a library of protein variants can be generated by combinatorial mutagenesis at the nucleic acid level such as, for example, by enzymatic ligation of a mixture of synthetic oligonucleotides. There is a large number of methods which can be used to prepare libraries of potential homologs from a degenerate oligonucleotide sequence. Chemical synthesis of a degenerate gene sequence can be carried out in an automatic DNA synthesizer, and the synthetic gene can then be ligated into a suitable expression vector. The use of a degenerate set of genes makes it possible to provide in a mixture all the sequences which code for the desired set of potential protein sequences. Methods for synthesizing degenerate oligonucleotides are known to a person skilled in the art (e.g. Narang, S. A. (1983) Tetrahedron 39:3; Itakura et al. (1984) Annu. Rev. Biochem. 53:323; Itakura et al., (1984) Science 198:1056; Ike et al. (1983) Nucleic Acids Res. 11:477).

Several techniques are known in the prior art for screening gene products in combinatorial libraries which have been prepared by point mutations or truncation, and for screening cDNA libraries for gene products having a selected property. These techniques can be adapted to the rapid screening of gene libraries which have been generated by combinatorial mutagenesis of homologs according to the invention. The most commonly used techniques for screening large gene libraries, which are subject to high-throughput analysis, comprise the cloning of the gene library into replicable expression vectors, transforming suitable cells with the resulting vector library and expressing the combinatorial genes under conditions under which detection of the desired activity facilitates isolation of the vector which codes for the gene whose product has been detected. Recursive ensemble mutagenesis (REM), a technique which increases the frequencies of functional mutants in the libraries, may be used in combination with the screening tests to identify homologs (Arkin and Yourvan (1992) PNAS 89:7811-7815; Delgrave et al. (1993) Protein Engineering 6(3):327-331).

Furthermore, a person skilled in the art is familiar with processes for generating functional mutants of the endoglucanases used herein by way of example.

Depending on the technique used, a person skilled in the art can introduce entirely random or else more targeted mutations into genes or else noncoding nucleic acid regions (which are for example important for regulating expression) and subsequently construct gene libraries. The methods of molecular biology which are required for this purpose are known to a person skilled in the art and described, for example, in Sambrook and Russell, Molecular Cloning, 3^{rd} Ed., Cold Spring Harbor Laboratory Press 2001.

Methods of modifying genes and thus of modifying the proteins encoded by them have long been known to a person skilled in the art, such as, for example,
site-specific mutagenesis, where individual or more nucleotides of a gene are replaced in a targeted manner (Trower M K (Ed.) 1996; In vitro mutagenesis protocols. Humana Press, New Jersey),
saturation mutagenesis where a codon for any amino acid may be replaced or added at any gene locus (Kegler-Ebo D M, Docktor C M, DiMaio D (1994) Nucleic Acids Res 22:1593; Barettino D, Feigenbutz M, Valcárel R, Stunnenberg H G (1994) Nucleic Acids Res 22:541; Barik S (1995) Mol Biotechnol 3:1),
error-prone polymerase chain reaction (error-prone PCR), where nucleotide sequences are mutated by erroneously working DNA polymerases (Eckert K A, Kunkel T A (1990) Nucleic Acids Res 18:3739);
the SeSaM method (Sequence Saturation Method), in which preferential substitutions are prevented by the polymerase. Schenk et al., Biospektrum, Vol. 3, 2006, 277-279
the passaging of genes in mutator strains, in which, for example, an increased mutation rate of nucleotide sequences takes place on account of defective DNA repair mechanisms (Greener A, Callahan M, Jerpseth B (1996) An efficient random mutagenesis technique using an E. coli mutator strain. In: Trower M K (Ed.) In vitro mutagenesis protocols. Humana Press, New Jersey), or
DNA shuffling, in which a pool of closely related genes is formed and digested and the fragments are used as templates for a polymerase chain reaction, in which mosaic genes of full length are finally produced by repeated strand separation and reannealing (Stemmer W P C (1994) Nature 370:389; Stemmer W P C (1994) Proc Natl Acad Sci USA 91:10747).

Using “directed evolution” (described, inter alia, in Reetz M T and Jaeger K-E (1999), Topics Curr Chem 200:31; Zhao H, Moore J C, Volkov A A, Arnold F H (1999), Methods for optimizing industrial enzymes by directed evolution, In: Demain A L, Davies J E (Ed.) Manual of industrial microbiology and biotechnology. American Society for Microbiology), a person skilled in the art can also generate functional mutants in a selective manner and also on a large scale. Here, in a first step, gene libraries of the respective proteins are initially produced, it being possible to employ, for example, the methods indicated hereinabove. The gene libraries are expressed in a suitable manner, for example by bacteria or by phage display systems.

The relevant genes of host organisms that express functional mutants with properties which largely correspond to the desired properties may be subjected to a further mutation cycle. The steps of mutation and selection or screening can be repeated iteratively until the functional mutants present possess the desired properties in an adequate measure. As a result of this iterative procedure, a limited number of mutations, such as, for example, 1 to 5 mutations, may be performed stepwise, and assessed and selected for their influence on the respective enzyme property. Then, the selected mutant may be subjected to a further mutation step in the same manner. The number of individual mutants to be studied may be significantly decreased thereby.

Suitable expression constructs can be employed in particular for the recombinant preparation of endoglucanases which can be used in accordance with the invention.

Subject matter of the invention are further expression constructs comprising, under the genetic control of regulatory nucleic acid sequences, a nucleic acid sequence which codes for an enzyme according to the invention; and vectors comprising at least one of these expression constructs.

According to the invention, an “expression unit” is understood as meaning a nucleic acid which has expression activity, which comprises a promoter as herein defined and which, after functional linkage to a nucleic acid to be expressed or a gene, will regulate the expression, in other words the transcription and the translation, of this nucleic acid or this gene. This is why it is also referred to in this context as a “regulatory nucleic acid sequence”. In addition to the promoter, further regulatory elements such as, for example, enhancers may be present.

According to the invention, an “expression cassette” or “expression construct” is understood as meaning an expression unit which is functionally linked to the nucleic acid to be expressed or the gene to be expressed. In contrast to an expression unit, an expression cassette, therefore, does not only comprise nucleic acid sequences which regulate transcription and translation, but also those nucleic acid sequences which are to be expressed as a protein as a result of transcription and translation.

Within the context of the invention, the terms “expression” or “overexpression” describe the production or increase of the intracellular activity of one or more enzymes in a microorganism which are encoded by the corresponding DNA. To this end, it is possible, for example, to introduce a gene into an organism, to replace an existing gene by a different gene, to increase the copy number of the gene(s), to use a strong promoter or to use a gene which encodes a corresponding enzyme with a high activity, and these measures can optionally be combined.

Preferably, such constructs according to the invention comprise a promoter 5′-upstream and a terminator sequence 3′-downstream of the respective coding sequence and, optionally, further customary regulatory elements, in each case operably linked to the coding sequence.

According to the invention, a “promoter”, a “nucleic acid with promoter activity” or a “promoter sequence” is understood as meaning a nucleic acid which, in functional linkage with a nucleic acid to be transcribed, regulates the transcription of this nucleic acid.

In this context, a “functional” or “operable” linkage is understood as meaning, for example, the sequential arrangement of one of the nucleic acids with promoter activity and a nucleic acid sequence to be transcribed and optionally further regulatory elements such as, for example, nucleic acid sequences which ensure the transcription of nucleic acids and, for example, a terminator in such a way that each of the regulatory elements can fulfill its function upon transcription of the nucleic acid sequence. A direct linkage in the chemical sense is not necessarily required for this purpose. Genetic control sequences such as, for example, enhancer sequences can also exert their function on the target sequence from positions which are located at a greater distance, or indeed from other DNA molecules. Preferred arrangements are those in which the nucleic acid sequence to be transcribed is positioned behind (i.e. at the 3′ end) of the promoter sequence so that the two sequences are covalently bonded to each other. In this context, the distance between the promoter sequence and the nucleic acid sequence to be expressed recombinantly may be less than 200 base pairs or less than 100 base pairs or less than 50 base pairs.

Further examples of regulatory elements which may be mentioned besides promoters and terminator are targeting sequences, enhancers, polyadenylation signals, selectable markers, amplification signals, replication origins and the like. Suitable regulatory sequences are described, for example, in Goeddel, Gene Expression Technology: Methods in Enzymology 185, Academic Press, San Diego, Calif. (1990).

Nucleic acid constructs according to the invention comprise, in particular, those in which the coding sequence has advantageously been operably or functionally linked to one or more regulatory signals for controlling, for example increasing, gene expression.

In addition to these regulatory sequences, the natural regulation of these sequences may still be present upstream of the actual structural genes and may optionally have been genetically modified so that the natural regulation has been switched off and the expression of the genes has been increased. However, the nucleic acid construct may also be simpler in construction, in other words no additional regulatory signals have been inserted upstream of the coding sequence and the natural promoter together with its regulation has not been removed. Instead, the natural regulatory sequence is mutated in such a way that regulation no longer takes place and gene expression is increased.

A preferred nucleic acid construct advantageously also comprises one or more of the already mentioned “enhancer” sequences functionally linked to the promoter, and these make possible an increased expression of the nucleic acid sequence. Also, it is possible to insert, at the 3′ end of the DNA sequences, additional advantageous sequences such as further regulatory elements or terminators. The nucleic acids according to the invention may be present in the construct as one or more copies. Further markers, such as antibiotic resistances or auxotrophism-complementing genes, may optionally additionally be present in the construct for selection onto the construct.

Examples of suitable regulatory sequences are present in promoters such as cos, tac, trp, tet, trp-tet, lpp, lac, lpp-lac, lacl^{q}, T7, T5, T3, gal, trc, ara, rhaP (rhaP_{BAD})SP6, lambda-P_{R} or BAD, in the lambda-P_{L} promoter which are advantageously employed in Gram-negative bacteria. Further advantageous regulatory sequences are present for example in the Gram-positive promoters amy and SPO2, in the yeast or fungal promoters ADC1, MFalpha, AC, P-60, CYC1, GAPDH, TEF, rp28, ADH. Artificial promoters for regulation may also be employed.

For expression, the nucleic acid construct is inserted into a host organism, advantageously a vector such as, for example, a plasmid or a phage, which makes possible the optimal expression of the genes in the host. In addition to plasmids and phages, vectors are also understood as meaning all the other vectors known to a person skilled in the art, that is to say, for example, viruses such as SV40, CMV, baculovirus and adenovirus, transposons, IS elements, phasmids, cosmids and linear or circular DNA. These vectors are amenable to autonomous replication in the host organism or to chromosomal replication. These vectors constitute a further embodiment of the invention.

Suitable plasmids are, for example, in E. coli pLG338, pACYC184, pBR322, pUC18, pUC19, pKC30, pRep4, pHS1, pKK223-3, pDHE19.2, pHS2, pPLc236, pMBL24, pLG200, pUR290, pIN-III¹¹³-B1, λgt11 or pBdCl, in Streptomyces pIJ101, pIJ364, pIJ702 or pIJ361, in Bacillus pUB110, pC194 or pBD214, in Corynebacterium pSA77 or pAJ667, in fungi pALS1, pIL2 or pBB116, in yeasts 2alphaM, pAG-1, YEp6, YEp13 or pEMBLYe23 or in plants pLGV23, pGHlac⁺, pBIN19, pAK2004 or pDH51. The abovementioned plasmids are a small selection of the plasmids which are possible. Further plasmids are well known to a person skilled in the art and can be found for example in the book Cloning Vectors (Eds. Pouwels P. H. et al. Elsevier, Amsterdam-New York-Oxford, 1985, ISBN 0 444 904018).

In a further configuration of the vector, the vector comprising the nucleic acid construct according to the invention or the nucleic acid according to the invention may also advantageously be introduced into the microorganisms in the form of a linear DNA and integrated into the genome of the host organism via heterologous or homologous recombination. This linear DNA may consist of a linearized vector, such as a plasmid, or else only of the nucleic acid construct or the nucleic acid according to the invention.

For an optimal expression of heterologous genes in organisms, it is advantageous to modify the nucleic acid sequences according to the specific “codon usage” used in the organism. The “codon usage” can be determined readily by computer evaluations of other, known genes of the organism in question.

An expression cassette according to the invention is prepared by fusing a suitable promoter to a suitable coding nucleotide sequence and a terminator or polyadenylation signal. To this end, one will employ customary recombination and cloning techniques as they are described, for example, in T. Maniatis, E. F. Fritsch and J. Sambrook, Molecular Cloning: A Laboratory Manual, Cold Spring Harbor Laboratory, Cold Spring Harbor, N.Y. (1989) and in T. J. Silhavy, M. L. Berman and L. W. Enquist, Experiments with Gene Fusions, Cold Spring Harbor Laboratory, Cold Spring Harbor, N.Y. (1984) and in Ausubel, F. M. et al., Current Protocols in Molecular Biology, Greene Publishing Assoc. and Wiley Interscience (1987).

For expression, the recombinant nucleic acid construct or gene construct is inserted into a suitable host organism, advantageously a host-specific vector, which makes possible an optimal expression of the genes in the host. Vectors are well known to a person skilled in the art and can be found, for example, in “Cloning Vectors” (Pouwels P. H. et al., Ed., Elsevier, Amsterdam-New York-Oxford, 1985).

It is possible with the aid of the vectors according to the invention to generate recombinant microorganisms which are for example transformed with at least one vector according to the invention and which can be employed for the production of the endoglucanases which can be used in accordance with the invention. Advantageously, the above-described recombinant constructs according to the invention are introduced into a suitable host system and expressed. In this context, it is preferred to use cloning and transfection methods known to a person skilled in the art, such as, for example, coprecipitation, protoplast fusion, electroporation, retroviral transfection and the like so as to express the abovementioned nucleic acids in the respective expression system. Suitable systems are described, for example, in Current Protocols in Molecular Biology, F. Ausubel et al., Ed., Wiley Interscience, New York 1997, or Sambrook et al. Molecular Cloning: A Laboratory Manual. 2^{nd} Ed., Cold Spring Harbor Laboratory, Cold Spring Harbor Laboratory Press, Cold Spring Harbor, N.Y., 1989.

Suitable recombinant host organisms for the nucleic acid according to the invention or the nucleic acid construct are, in principle, all prokaryotic or eukaryotic organisms. It is advantageous to use microorganisms such as bacteria, fungi or yeasts as host organisms. It is advantageous to use Gram-positive or Gram-negative bacteria, preferably bacteria from the families Enterobacteriaceae, Pseudomonadaceae, Rhizobiaceae, Streptomycetaceae or Nocardiaceae, especially preferably bacteria from the genera Escherichia, Pseudomonas, Streptomyces, Nocardia, Burkholderia, Salmonella, Agrobacterium, Clostridium or Rhodococcus. The genus and species Escherichia coli is very especially preferred. Further advantageous bacteria can additionally be found in the group of the alpha-proteobacteria, beta-proteobacteria or gamma-proteobacteria.

In this context, the host organism(s) according to the invention preferably comprises/comprise at least one of the nucleic acid sequences which code for an endoglucanase, nucleic acid constructs or vectors which code for an enzyme with endoglucanase activity as defined hereinabove.

Depending on the host organism, the organisms used in the process according to the invention are grown or cultured in a manner known to a person skilled in the art. As a rule, microorganisms are grown in a liquid medium which comprises a carbon source, usually in the form of sugars, a nitrogen source, usually in the form of organic nitrogen sources such as yeast extract or salts such as ammonium sulfate, trace elements such as iron, manganese, magnesium salts and optionally vitamins, at temperatures of between 0° C. and 100° C., preferably between 10° C. and 60° C., while passing in oxygen. In this context, the pH of the liquid medium may be maintained at a fixed value, i.e. it may or may not be regulated during culturing. Culturing can take place batchwise, semibatchwise or continuously. Nutrients can be provided at the beginning of the fermentation or fed in semicontinuously or continuously.

For the recombinant production of endoglucanases which can be employed in accordance with the invention or of functional biologically active fragments thereof, a microorganism which produces this enzyme is cultured, the expression of the enzyme is optionally induced, and the enzyme is isolated from the culture. The polypeptides can thus also be produced on an industrial scale, if this is desired.

The microorganisms which have been produced in accordance with the invention can be grown continuously or discontinuously by the batch method, the fed-batch method or the repeated fed-batch method. An overview of known culture methods can be found in the textbook by Chmiel (BioprozeStechnik 1. Einfuhrung in die Bioverfahrenstechnik (Gustav Fischer Verlag, Stuttgart, 1991) [Bioprocess engineering 1. Introduction to bioprocess technology]) or in the textbook by Storhas (Bioreaktoren and periphere Einrichtungen [Bioreactors and peripheral units] (Vieweg Verlag, Braunschweig/Wiesbaden, 1994)).

The culture medium to be used must suitably meet the needs of the strains in question. Descriptions of culture media for a variety of microorganisms can be found in the manual “Manual of Methods for General Bacteriology” of the American Society for Bacteriology (Washington D. C., USA, 1981).

These media which can be employed in accordance with the invention usually comprise one or more carbon sources, nitrogen sources, inorganic salts, vitamins and/or trace elements.

Preferred carbon sources are sugars such as mono-, di- or polysaccharides. Examples of very good carbon sources are glucose, fructose, mannose, galactose, ribose, sorbose, ribulose, lactose, maltose, sucrose, raffinose, starch or cellulose. Sugars may also be added to the media via complex compounds, such as molasses, or other by-products of sugar refining. It may also be advantageous to add mixtures of various carbon sources. Other possible carbon sources are oils and fats such as, for example, soya oil, sunflower oil, peanut oil and coconut fat, fatty acids such as, for example, palmitic acid, stearic acid or linoleic acid, alcohols such as, for example, glycerol, methanol or ethanol, and organic acids such as, for example, acetic acid or lactic acid.

Nitrogen sources are usually organic or inorganic nitrogen compounds or materials comprising these compounds. Examples of nitrogen sources encompass ammonia gas or ammonium salts such as ammonium sulfate, ammonium chloride, ammonium phosphate, ammonium carbonate or ammonium nitrate, nitrates, urea, amino acids or complex nitrogen sources such as corn steep liquor, soya meal, soya protein, yeast extract, meat extract and others. The nitrogen sources can be used individually or as a mixture.

Inorganic salt compounds which may be present in the media encompass the chloride, phosphorus or sulfate salts of calcium, magnesium, sodium, cobalt, molybdenum, potassium, manganese, zinc, copper and iron.

Inorganic sulfur-containing compounds such as, for example, sulfates, sulfites, dithionites, tetrathionates, thiosulfates, sulfides, but also organic sulfur compounds, such as mercaptans and thiols, may be used as the sulfur source.

Phosphoric acid, potassium dihydrogenphosphate or dipotassium hydrogenphosphate or the corresponding sodium-containing salts may be used as phosphorus source.

Sequestrants may be added to the medium in order to maintain the metal ions in solution. Particularly suitable sequestrants encompass dihydroxyphenols, such as catechol or protocatechuate, or organic acids such as citric acid.

Usually, the fermentation media employed in accordance with the invention also comprise other growth factors such as vitamins or growth promoters, which include, for example, biotin, riboflavin, thiamine, folic acid, nicotinic acid, panthothenate and pyridoxine. Growth factors and salts are frequently obtained from complex media components such as yeast extract, molasses, corn steep liquor and the like. Moreover, suitable precursors may be added to the culture medium. The exact composition of the compounds in the media depends greatly on the experiment in question and will be decided individually for each individual case. Information on the optimization of media can be found in the textbook “Applied Microbiol. Physiology, A Practical Approach” (Eds P. M. Rhodes, P. F. Stanbury, IRL Press (1997) pp. 53-73, ISBN 0 19 963577 3). Growth media can also be obtained from commercial sources, such as Standard 1 (Merck) or BHI (brain heart infusion, DIFCO) and the like.

All of the components of the media are sterilized, either by means of heat (20 minutes at 1.5 bar and 121° C.) or by filter sterilization. The components may be sterilized either together or, if appropriate, separately. All of the components of the media may be present at the beginning of the fermentation or else be added continuously or batchwise, as desired.

The culture temperature is normally between 15° C. and 45° C., preferably 25° C. to 40° C., and can be kept constant during the experiment or else be varied. The pH of the medium should be in the range of from 5 to 8.5, preferably around 7.0. The pH for the fermentation can be controlled during the fermentation by addition of basic compounds such as sodium hydroxide, potassium hydroxide, ammonia or ammonia water, or acidic compounds such as phosphoric acid or sulfuric acid. Antifoam agents such as, for example, fatty acid polyglycol esters may be employed to control foam development. To maintain plasmid stability, suitable selectively acting substances such as, for example, antibiotics may be added to the medium. To maintain aerobic conditions, oxygen or oxygen-containing gas mixtures, for example ambient air, are passed into the culture. The culture temperature is normally 20° C. to 45° C. and. The culture is continued until a maximum of the desired product has formed. This aim is normally achieved within 10 hours to 160 hours.

Thereafter, the fermentation broth is processed further. Depending on what is required, all or some of the biomass may be removed from the fermentation broth by separation methods such as, for example, centrifugation, filtration, decanting or a combination of these methods, or else be left completely in said broth.

If the polypeptides are not secreted into the culture medium, the cells may also be disrupted and the product may be obtained from the lysate by customary protein isolation methods. The cells can optionally be disrupted by high-frequency ultrasound, by high pressure such as, for example, in a French pressure cell, by osmolysis, by the action of detergents, lytic enzymes or organic solvents, by homogenizers or by a combination of several of the abovementioned methods.

The polypeptides may be purified using known chromatographic techniques, such as molecular sieve chromatography (gel filtration), such as Q-Sepharose chromatography, ion exchange chromatography and hydrophobic chromatography, and also using other common methods such as ultrafiltration, crystallization, salting out, dialysis and native gel electrophoresis. Suitable methods are described for example in Cooper, T. G., Biochemische Arbeitsmethoden [Biochemical working methods], Verlag Walter de Gruyter, Berlin, N.Y. or in Scopes, R., Protein Purification, Springer Verlag, New York, Heidelberg, Berlin.

To isolate the recombinant protein, it may be advantageous to use vector systems or oligonucleotides which extend the cDNA by specific nucleotide sequences and thus encode modified polypeptides or fusion proteins which serve, for example, the purpose of simpler purification. Such modifications which are suitable are, for example, what are known as “tags”, which act as anchors, such as, for example, the modification known as hexa-histidine anchor or epitopes capable of being recognized by antibodies as being antigens (described, for example, in Harlow, E. and Lane, D., 1988, Antibodies: A Laboratory Manual. Cold Spring Harbor (N.Y.) Press). These anchors may serve to attach the proteins to a solid support, such as, for example, a polymer matrix which may be packed into, for example, a chromatography column, or for attaching the proteins to a microtiter plate or any other support.

At the same time, these anchors can also be used for the identification of the proteins. For identifying the proteins, customary markers, such as fluorescent dyes, enzyme markers that, after reaction with a substrate, form a detectable reaction product, or radioactive markers, can moreover be used alone or in combination with the anchors for derivatization of the proteins.

The endoglucanase can be employed in free or immobilized form. An immobilized enzyme is understood as meaning an enzyme which is fixed to an inert support. Suitable carrier materials and the enzymes immobilized thereon are known from EP-A-1149849, EP-A-1 069 183 and DE-OS 100193773, and from the literature references cited therein. Reference is made to the relevant disclosure of these documents in its entirety. Suitable carrier materials include, for example, clays, clay minerals such as kaolinite, diatomaceous earth, perlite, silicon dioxide, aluminum oxide, sodium carbonate, calcium carbonate, cellulose powder, anion exchanger materials, synthetic polymers such as polystyrene, acrylic resins, phenol/formaldehyde resins, polyurethanes and polyolefins, such as polyethylene and polypropylene. For the production of the supported enzymes, the carrier materials are usually employed in a finely-divided particulate form, with porous forms being preferred. The particle size of the carrier material is usually not more than 5 mm, in particular not more than 2 mm (particle-size distribution curve). Similarly, when employing the endoglucanase as a whole-cell catalyst, a free or immobilized form may be selected. Carrier materials are, for example, calcium alginate and carrageenan. Enzymes as well as cells may also be crosslinked directly with glutaraldehyde (crosslinking to CLEAs). Corresponding and further immobilization techniques are described, for example, in J. Lalonde and A. Margolin “Immobilization of Enzymes” in K. Drauz and H. Waldmann, Enzyme Catalysis in Organic Synthesis 2002, Vol. III, 991-1032, Wiley-VCH, Weinheim.

### 3.2 Cellooligomer Production

The cellooligomers are produced by single or multiple enzymatic hydrolysis with the aid of endoglucanases, optionally in combination with a treatment of the cellulose with ionic liquids and/or mechanical treatment. The optimal order can be determined by a person skilled in the art by simple preliminary experiments.

a) Cellulose Treatment with Ionic Liquids

To this end, the cellulose is suspended for example in cold EMIM Ac. To this end, for example 0.02 g of cellulose is heated per ml of EMIM Ac until the liquid turns clear. The cellulose is then precipitated with water. The precipitated cellulose is separated from the ionic liquid and the water employed for the precipitation by a vacuum filtration. The cellulose is then washed with water.

### b) Mechanical Cellulose Treatment

10% (w/w) of cellulose is suspended in distilled water. A ball mill (for example Beadbeater, Biospec Products) is prepared with 1 mm glass beads as per instruction, filled with the cellulose suspension and ground. After cooling, the cellulose is ground again. This process is repeated several times on ice. Thereafter, the cellulose is eluted from the glass beads using distilled water until the elution water runs clear. The eluted suspension is collected and then centrifuged. The water is decanted off from the pretreated cellulose and the cellulose is employed in the enzymatic hydrolysis.

### c) Enzymatic Cellulose Hydrolysis

Freshly pretreated cellulose is employed for the enzymatic hydrolysis. A suitable buffer (acetate buffer, phosphate buffer or Tris buffer, 0.01 to 0.25 M, pH 4 to 7) is prepared for the hydrolysis, for example 0.1 M Na acetate buffer pH 4.5 and 0.1 M Na—K phosphate buffer pH 6. The optimal buffer concentration and pH can be established by carrying out just a few preliminary experiments.

The cellulases are dissolved in buffer at the desired concentration. The cellulase solution is added to the weighed cellulose and mixed in a vortex mixer for some seconds. Unless otherwise specified, approximately 10 mg (dry weight) of cellulose in 2 ml Eppendorf reaction vessels together with 1 ml of cellulase solution are used for the hydrolysis, and these are incubated for the desired period of time in a thermomixer at 40° C. and 800 min⁻¹. To terminate the reaction, the samples are spun down in a tabletop centrifuge at 14 000 min⁻¹ for 3 min. After some routine preliminary experiments, a corresponding procedure with larger batches is also possible.

### c) Further Enzymatic Hydrolysis

To further degrade incompletely hydrolyzed cellulose, cellulose which has already been hydrolyzed can be retreated with ionic liquid, and the treated cellulose can be subjected to another enzymatic hydrolysis. This is carried out as described hereinabove after pretreatment with ionic liquid, likewise as described hereinabove.

The invention will now be illustrated in greater detail in the experimental part with reference to the following specific embodiments.

### 3.3 Applications

Fields of application—without wishing to limit them by enumeration—are for example in the field of fiber reinforcement or the modification of surface-active substances or formulas (antifoams, rheology-modifying agents and the like).

### Experimental Part

### I. Materials

### Celluloses:

In accordance with the invention, various cellulose substrates with different property profiles were used. The cellulose substrates differ in respect of their degree of polymerization or their chain-length distribution and their degree of crystallinity and the available surface. Table 1 gives an overview of the substrates used.

TABLE 1
Cellulose substrates used, and their properties

| | | | | | Particle | | |
|---|---|---|---|---|---|---|---|
| | | Product | | Crl | diameter | | |
| Substrate | Manufacturer | number | Contaminations | [%] | [μm] | DP_{W} | DP_{N} |
| α-Cellulose | Sigma | C8002- | Xylan | 64 | 68.77 | 370 | 70 |
| | | 1KG | | | | | |
| Avicel PH-101 | Fluka | 11365 | none | 82 | 43.82 | 155 | 55 |
| Sigmacell Typ 101 | Sigma | S6790- | none | ≈0 | 15.86 | 295 | 160 |
| | | 500G | | | | | |

DP_{W} and DP_{N} was determined via the GPC_{w} described herein.

The degree of polymerization and the chain length distribution of a cellulose substrate are determined by GPC. To carry out this measurement, the samples were derivatized by existing methods and subsequently measured by GPC [Rinaldi et al. Angewandte Chemie-International Edition, 47(42):8047-8050, 2008, Rinaldi et al., Chemsuschem, 3(2):266-276, 2010]. The samples analyzed in accordance with the invention need no longer be derivatized for the GPC measurement due to an alternative GPC method [Engel et al. 2012, Biotechnology for Biofuels 5:77]. Table 1 shows the determined degree of polymerization of the substrates as per the GPC measuring system used in accordance with the invention.

### Enzymes:

TABLE 2
Overview of the cellulases employed (by Megazyme Inc.)

| | | | Temperature | Molecular |
|---|---|---|---|---|
| | | pH | optima | weight |
| Cellulase type | Species | optima | [° C.] | [kDa] |
| Endoglucanase | A. niger | 4.5 | 60 | 27 |
| | B. amyloliquefaciens | 6.0 | 60 | 34.3 |
| | T. maritima | 6.0 | 80 | 38.2 |
| | T. emersonii | 4.5-4.6 | 70 | 37 |
| | T. longibrachiatum | 4.5-5.0 | 70 | 57.25 |
| β-Glucosidase | Agrobacterium sp. | 6.5-7.0 | 50 | 52.2 |
| | A. niger | 4.0 | 70 | 121 |

### II. Apparatus

TABLE 3
Instrumentation of organic GPC

| Type of instrument | Manufacturer | Model |
|---|---|---|
| GPC columns | PSS | GRAL 2x10000 Å & |
| | | 1x30 Å |
| GPC oven | Techlab | K7 |
| HPLC pump | Agilent | 1500 series |
| and autosampler | | |
| LS detector | WYATT | Wyatt DAWN HELIOS 8 |
| Recycling system | WYATT | Orbit |
| RI detector | WYATT | optilab rEX |
| Software | Agilent Technologies | ASTRA Version 5.3.5.14 |

TABLE 4
Instrumentation of aqueous GPC

| Type of instrument | Manufacturer | Model |
|---|---|---|
| GPC columns | PSS | Suprema analytical |
| | | 2x100 Å & 1x30 Å |
| GPC oven | TOSOH | ECO HLC8320GPC |
| HPLC pump and autosampler | | |
| Recycling system | | |
| RI detector | | |
| Software | Software | WinGPC Unity PSS |

TABLE 5
HPLC instrumentation

| Type of instrument | Manufacturer | Model |
|---|---|---|
| Detector | Shodex | RI-71 UVD340S |
| | | (210, 220, 230 and 300) |
| Column oven | Dionex | STH 585 |
| Autosampler | Gilson | 232XL |
| Pump | Dionex | P580 |
| HPLC column | CS-Chromatographie | Organic Acid-Resin 250 × 8 mm |
| | Service | including precolumn |
| Software | Dionex | Chromeleon analytical software |

### III. Methods

1. Pretreatment of Cellulose with Ionic Liquids

The cellulose is suspended in cold EMIM Ac. To this end, 0.75 g of cellulose per 50 ml of EMIM Ac is heated to 80° C. for at least 40 min until the liquid turns clear. Thereafter, the cellulose is precipitated with 400 ml of water. The precipitated cellulose is separated from the ionic liquid and the water employed for the precipitation via vacuum filtration, in which a cellulose acetate filter with mesh size 0.2 μm is employed. Thereafter, the cellulose is washed 3 times with 500 ml of water.

### 2. Mechanical Cellulose Pretreatment

10% (w/w) of cellulose is suspended in distilled water. The ball mill (Beadbeater, Biospec Products) is prepared with 1 mm glass beads as per instruction, filled with the cellulose suspension and ground for 3 min. Thereafter, the ball mill must cool down for 3 min until the cellulose can be ground again. This procedure is repeated five times on ice. Thereafter, the cellulose is eluted from the glass beads with distilled water until the elution water runs clear. The eluted suspension is collected and then centrifuged for 3 min at 4000 min⁻¹ (Rotina 35R). The water is decanted off from the pretreated cellulose and the cellulose is employed in the enzymatic hydrolysis.

### 3. Enzymatic Cellulose Hydrolysis

Freshly pretreated cellulose is employed for the enzymatic hydrolysis. Since the cellulose is not dried after the pretreatment, the wet weight must be converted into dry weight. In this context, it is not possible to determine the dry weight beforehand by drying some of the pretreated cellulose since this takes several days. To calculate the swelling factor, one therefore assumes 5% cellulose loss as the result of the pretreatment. The wet weight of the pretreated cellulose is determined and divided by 95% of the weight employed for the pretreatment. The swelling factor determined then serves for calculating the required amount of pretreated moist cellulose. Since the wet weight may vary after the pretreatment, this factor has to be redetermined after each pretreatment. 0.1 M Na acetate buffer pH 4.5 and 0.1 M Na—K phosphate buffer pH 6 are prepared for the hydrolysis.

The A. niger, T. longibrachiatum and T. emersonii endoglucanases and the A. niger β-glucosidase are dissolved in Na acetate buffer at the desired concentration. The T. maritima and B. amyloliquefaciens endoglucanases are dissolved in Na—K phosphate buffer at the desired concentration. The cellulase solution is added to the weighed cellulose and mixed in a vortex mixer for a few seconds. Unless otherwise specified, 10 mg (dry weight) of cellulose in 2 ml Eppendorf reaction vessels together with 1 ml of cellulase solution are used for the hydrolysis, and these are incubated for the desired period of time in a thermomixer at 40° C. and 800 min⁻¹. To terminate the reaction, the samples are spun down in a tabletop centrifuge at 14 000 min⁻¹ for 3 min, and the supernatant is removed and transferred into another Eppendorf reaction vessel. The pellet and the supernatant are frozen at −80° C. and can be analyzed at a later point in time.

4. Second Enzymatic Hydrolysis Following Renewed Pretreatment with Ionic Liquid (IL Restart)

To study incomplete cellulose hydrolysis owing to structural properties or changes in the cellulose, such as, for example, crystallinity or recrystallinity, cellulose which has already been hydrolyzed is retreated with ionic liquid, and the treated cellulose is again subjected to enzymatic hydrolysis. The IL restart is carried out after the hydrolysis of cellulose pretreated with ionic liquids. 10 U/ml endoglucanase and 3 U/ml β-glucosidase in the respective buffers are employed for both hydrolysis steps.

Since freshly hydrolyzed cellulose is employed for the IL restart, the cellulose loss after hydrolysis is calculated. To this end, the cellulose loss after the hydrolysis of the five endoglucanases employed must be determined beforehand (see hereinbelow). The determined cellulose loss is used to calculate the amount of cellulose after hydrolysis (dry weight). The pellet obtained after hydrolysis is dissolved in EMIM Ac at 80° C. for 2 h. The required amount of EMIM Ac depends on the amount and the moisture content of the cellulose because the water present in the cellulose samples reduces the cellulose dissolution capacity of EMIM Ac. Thereafter, the cellulose is precipitated from EMIM Ac by adding water. The cellulose is washed three times with water. To separate off the liquid, the cellulose is centrifuged for 3 min at 4000 min⁻¹ (Rotina 35R) and the liquid is subsequently decanted off. The wet weight of the cellulose samples is determined by weighing. The determined wet weight of the samples and the calculated dry weights after hydrolysis are used to determine the swelling factor of the samples. The cellulose purified by the cellulase in this method is subsequently employed for a second hydrolysis. To determine the wet weights of the cellulose, the previously determined swelling factor is used.

### IV. Analytical Methods

### 1. Organic Gel Permeation Chromatography

Organic gel permeation chromatography (GPCₒ) is used to determine the chain length distribution of cellulose samples. To carry out the GPOₒ, the cellulose samples from the hydrolysis are lyophilized, and the lyophilizate is subsequently dissolved in DMF/19% (v/v) EMIM Ac at 80° C. The dissolved cellulose is filtered through a 0.1 μm PT-FE filter and the filtrate transferred into HPLC glass vessels. A GPOₒ system with the abovementioned instrumentation (Tab. 3) is used for the analysis under the following conditions.

| Operating parameter | Configuration |
|---|---|
| Mobile phase | DMF/10% EMIM Ac (v/v) |
| Operating temperature | 50° C. |
| Flow rate | 0.5 ml/min |

The concentration is detected via an RI detector. The molar mass of the cellulose chains is determined via a scattered light detector. The detection limit of cellooligomers is at a degree of polymerization of approx. 10. Both the DP_{W} and the DP_{N} are determined with the aid of ASTRA Software.

### 2. Aqueous Gel Permeation Chromatography

Aqueous gel permeation chromatography (GPC_{w}) will be used to determine the composition of the sugars in the aqueous hydrolysis supernatant. For the aqueous GPC, the supernatants from the hydrolysis are filter-sterilized (0.2 μm) and transferred into HPLC glass vessels. A GPC system with the components of Table 4 is used for the analysis under the following conditions.

| Operating parameter | Configuration |
|---|---|
| Mobile phase | 50 mM Na-Ac buffer pH 5 |
| Operating temperature | 40° C. |
| Flow rate | 1 ml/min |

Glucose and cellooligomers (cellobiose [C2] to cellohexaose [C6]) from Megazyme (Ireland) are used for the calibration. The retention time is 27 to 31.5 min. The analyte is detected via an RI detector.

### 3. Dry Matter Determination

For a posteriori determination of the precise amount of cellulose employed in the hydrolysis, the dry matter of the pretreated cellulose is determined. To this end, Eppendorf reaction vessels are labeled and dried for 3 days at 80° C. After recording the weight, the Eppendorf reaction vessel is filled with a specific amount of wet cellulose and the weight is recorded. Thereafter, the filled Eppendorf reaction vessel is dried at 80° C. After drying, the Eppendorf reaction vessel is weighed once more. The swelling factor will be determined from the empty wet, the wet weight and the dry weight. Since freshly pretreated cellulose is used for all experiments, the swelling factor determined merely serves to subsequently verify the values calculated for the hydrolysis experiments.

Swelling factor=wet weight/dry weight

### 4. Cellulose Loss Determination

The cellulose loss determination allows the determination of the amount of cellulose which is broken down into soluble sugars and cellooligomers. To determine the cellulose loss, 30 mg of cellulose dry matter are hydrolyzed by the above-described method.

However, the cellulose is not separated from the buffer by centrifugation. The weight of a cellulose acetate filter of pore size 0.2 μm is recorded, and the hydrolyzed cellulose suspension is filtered through the filter. The filter together with the hydrolyzed cellulose is washed 3 times with 10 ml of distilled water and dried for 3 days at 80° C. After drying, the cellulose acetate filter together with the dried cellulose is weighed, and the weight is recorded. The cellulose loss can be determined from the weight difference between the cellulose employed for hydrolysis and the weight after drying of the cellulose.

The weight tends to be determined as too high because any cellulase adsorbed onto the cellulose is not removed by this method. In the case of unhydrolyzed cellulose, the cellulase which may be adhering amounts to between 0.8% and 2.3%. Owing to the production of glucose and soluble cellooligomers, the amount of cellulases increases during hydrolysis in relation to the insoluble cellulose. If the cellulose is reduced by 70%, the amount of cellulase which may adhere thereto attains a maximum 7.6%. The real loss of cellulose as a result of the formation of soluble sugars and cellooligomers is therefore slightly higher than what is determined by this method.

### 5. High-Performance Liquid Chromatography

High-performance liquid chromatography (HPLC) allows the concentration of the glucose and cellobiose in the aqueous supernatant of the hydrolysis mixture to be determined. To carry out the HPLC, the supernatants from the hydrolysis are filter-sterilized and transferred into HPLC glass vessels. An HPLC (Table 5) under the following conditions is used for the analysis:

| Operating parameter | Configuration |
|---|---|
| Mobile phase | 5 mM H₂SO₄ |
| Operating temperature | 60° C. |
| Flow rate | 0.6 ml/min |

### 6. 4-Hydroxybenzoic Acid Hydrazide Assay

4-Hydroxybenzoic acid hydrazide assay (PAHBAH assay) allows the molar concentration of the reducing sugars in the aqueous supernatant of the hydrolysis mixture to be determined. The reducing soluble sugars react with the PAHBAH reagent to give a yellow dye which can be measured photometrically at 410 nm. For calibration purposes, glucose calibration series are established with the used buffers at a concentration range of from 0.025 g to 0.5 g/I.

Two reagents (A and B), whose compositions are listed in the table hereinbelow, must be prepared for the PAHBAH assay. Before carrying out the PAHBAH assay, the working reagent is prepared from reagents A and B (ratio 1:10). The former will only keep for a few hours and should therefore be prepared immediately before carrying out the PAHBAH assay. The samples to be assayed and the calibration solutions are mixed with the working reagent in the ratio 1:3 and 1:5 and incubated at 100° C. for 15 min. After the samples and standard solutions have cooled to room temperature, they are measured at 410 nm in a photometer. The amount of soluble reducing sugars and cellooligomers can be determined in mol/ml with the aid of the calibration line determined by linear regression.

| Reagent | Substance | Mass/volume |
|---|---|---|
| Reagent A | p-Hydroxybenzoic | 5 g |
| | acid hydrazide | |
| | Water | 30 ml |
| | HCl (conc.) | 5 ml |
| | Water | make up to 100 ml |
| Reagent B | Trisodium citrate | 12.45 g |
| | Water | 500 ml |
| | Calcium chloride | 1.1 g |
| | Sodium hydroxide | 20 g |
| | Water | make up to 11 |

### 7. Sodium Dodecyl Sulfate Polyacrylamide Gel Electrophoresis

A sodium dodecyl sulfate polyacrylamide gel electrophoresis (SDS gel electrophoresis) with an SDS gel system from Life Technologies (California, USA) was used to verify the purity of the enzymes used.

### Conversion Examples

To prepare cellooligomers with a size near the solubility in water, experiments with the three cellulose substrates Avicel, α-cellulose and Sigmacell and with five endoglucanases and two β-glucosidases were first carried out. Before the beginning of the experiments, the enzymes employed were examined for purity, and the celluloses employed were examined for size distribution after swelling in the respective reaction buffer. Unless otherwise specified, an IL pretreatment was carried out first and an enzymatic hydrolysis was carried out subsequently for all enzymatic hydrolyses. The three celluloses were first hydrolyzed with all endoglucanases for 2 h. Thereafter, the reactions of the three endoglucanases with the lowest production of glucose and soluble cellooligomers were studied in the following experiments.

### Example 1

### GPCₒ of the Cellulose Substrates Avicel, α-Cellulose and Sigmacell

For carrying out the GPCo of the three enzymatically non-hydrolyzed cellulose substrates Avicel, α-cellulose and Sigmacell, samples of these three substrates were incubated in buffer for 1 day at 40° C. Thereafter, they were prepared for the GPCo analogously to the hydrolysis samples and then analyzed by GPCo.

For Avicel, the determination of the chain length distribution gave the same result for the acetate buffer and the phosphate buffer. The chain length distribution of α-cellulose and of Sigmacell samples which had been incubated in phosphate buffer show a proportional shift of the curve towards longer chains. The cause of this observation is unclear. The samples which had been incubated in acetate buffer are not shifted towards longer chain lengths in comparison with samples which had not been incubated in buffer. In what follows, substrate samples which had previously been incubated in acetate buffer and subsequently lyophilized are used as reference for showing the chain length distribution of α-cellulose and Sigmacell without enzymatic degradation.

### Example 2

### Hydrolysis of Avicel by Endoglucanases from A. niger, B. amyloliquefaciens, T. maritima, T. longibrachiatum and T. emersonii

The hydrolysis of Avicel by endoglucanases from A. niger, B. amyloliquefaciens and T. maritima was analyzed by GPCo.

A. niger

Composition of the hydrolysis mixture:

10 mg/ml Avicel,

10 U/ml A. niger endoglucanase,

3 U/ml A. niger β-glucosidase,

40° C., 0.1 M acetate buffer pH 4.5

Result: The chain length distribution of the sample without endoglucanase is between 10 and 1000 glucose units, with a maximum at 250. After 5 min hydrolysis with the A. niger endoglucanase, a shift of the upper end of the chain length distribution by 700 glucose units towards shorter chain lengths is observed. The chain length distribution after this reaction time is between 10 and 300 glucose units. The maximum of the distribution is at 90 glucose units. During the time between 5 min and 24 h, the upper end of the chain length distribution shifts by 100 glucose units towards shorter chain lengths. After 24 h, the curve is between 10 and 200 glucose units and has a maximum at 70 glucose units.

B. amyloliquefaciens

Composition of the hydrolysis mixture:

10 mg/ml Avicel,

10 U/ml B. amyloliquefaciens endoglucanase,

3 U/ml Agrobacterium sp. β-glucosidase,

40° C., 0.1 M phosphate buffer pH 6

Result: The chain length distribution of the sample without endoglucanase is between 10 and 700 glucose units, with a maximum at 250. For the hydrolysis of Avicel by B. amyloliquefaciens endoglucanase, too, a shift of the upper end of the chain length distribution towards shorter chains can be observed after 20 min. The chain length distribution of the 2-h sample is shifted towards shorter chain lengths by 150 glucose units in comparison with the 20-min sample. The curves of the samples between 2 h and 24 h are congruent. Accordingly, the substrate is not broken down any further. After hydrolysis for 24 h, a chain length distribution which is between 10 and 200 glucose units and has a maximum at approximately 65 glucose units is attained.

T. maritima

Composition of the hydrolysis mixture:

10 mg/ml Avicel,

10 U/ml T. maritima endoglucanase,

3 U/ml Agrobacterium sp. β-glucosidase,

40° C., 0.1 M phosphate buffer pH 6

The GPCo measurement of the sample without endoglucanase shows a chain length distribution of 10 to 700 glucose units. During the first 5 min of the hydrolysis of Avicel by T. maritima endoglucanase, a shift of the upper end of the chain distribution towards shorter chains is observed. This is analogous to the hydrolyses of Avicel by the A. niger and B. amyloliquefaciens endoglucanases. The 5-min sample shows a chain length distribution of between 15 and 300 glucose units. Until the end of the experiment after 24 h, the upper end of the chain length distribution continues to shift towards shorter chain lengths. After this reaction time, the chain length distribution is between 15 and 300 glucose units, with a maximum at 90 glucose units.

T. longibrachiatum

Composition of the hydrolysis mixture:

10 mg/ml Avicel,

10 U/ml T. longibrachiatum endoglucanase,

3 U/ml A. niger β-glucosidase,

40° C., 0.1 M acetate buffer pH 4.5

In comparison with the Avicel hydrolyses by the A. niger, B. amyloliquefaciens and T. maritima endoglucanases which have already been mentioned, the hydrolysis of Avicel by the T. longibrachiatum endoglucanase proceeds slower.

The upper end of the chain length distribution for the abovementioned endoglucanases after 5 min hydrolysis is between 300 and 400 glucose units. Only after 20 min is this the case for the T. longibrachiatum endoglucanase. However, after 24 h, the chain length distribution of 10 to 200 glucose units and a maximum at 60 glucose units is comparable to the corresponding chain length distributions of the A. niger, B. amyloliquefaciens and T. maritima endoglucanases.

T. emersonii

Composition of the hydrolysis mixture:

10 mg/ml Avicel,

10 U/ml T. emersonii endoglucanase,

3 U/ml A. niger β-glucosidase,

40° C., 0.1 M acetate buffer pH 4.5

The chain length distribution of the sample without endoglucanase is between 10 and 700 glucose units, with a maximum of 250 glucose units. In the hydrolysis of Avicel by the T. emersonii endoglucanase, the upper end of the chain length distribution shifts during the first 4 h of the hydrolysis towards shorter chain lengths. After 4 h, the curve of the chain length distribution is between 15 and 200 glucose units, with a maximum at 70 glucose units.

### SUMMARY

Using the five endoglucanases used, it is possible to achieve, after 24 h of hydrolysis, a shift of the upper end of the chain length distribution towards shorter chain lengths. The original course of the chain length distribution narrows as the hydrolysis increases. The lower end of the chain length distribution for all endoglucanases is in the range of between 7 and 15 glucose units. Thus, an endo-activity can be detected for all endoglucanases.

### Example 3

### Determination of the Degree of Polymerization DP of Enzymatically Hydrolyzed Avicel

GPC analyses were carried out to determine the DP of enzymatically hydrolyzed Avicel (of example 2).

### Sample Composition GPCo:

2 mg/ml cellulose lyophilizate in DMF/19% EMIM Ac (v/v)

Mobile phase: DMF/10% EMIM Ac (v/v), 50° C.

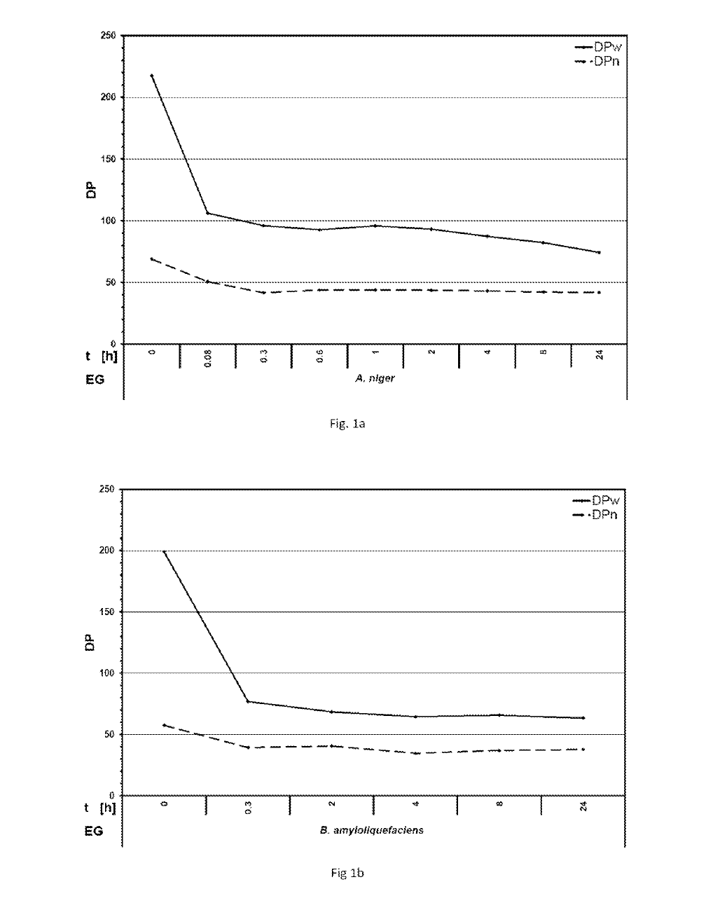

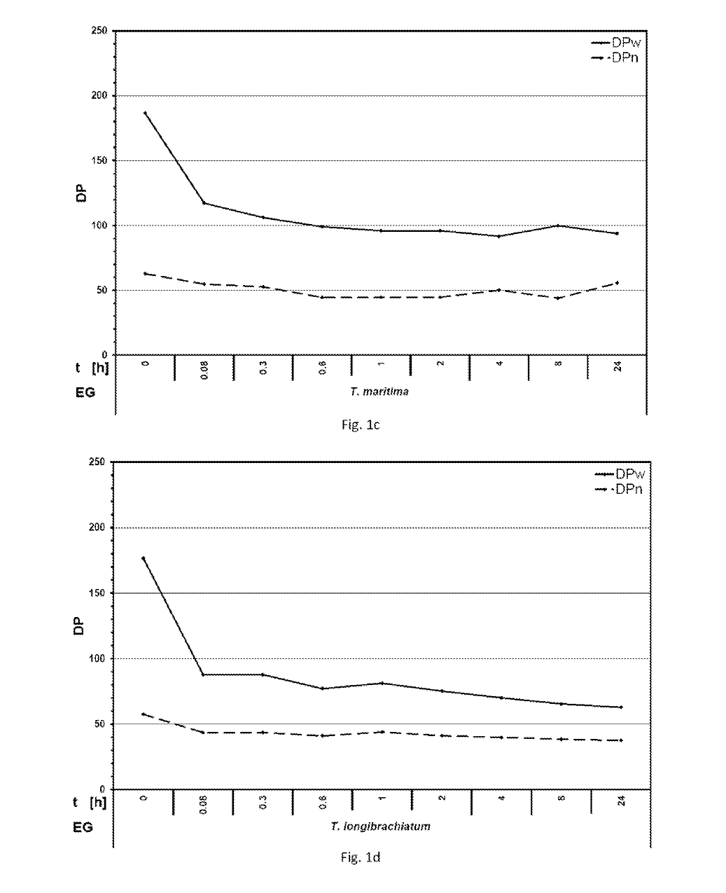

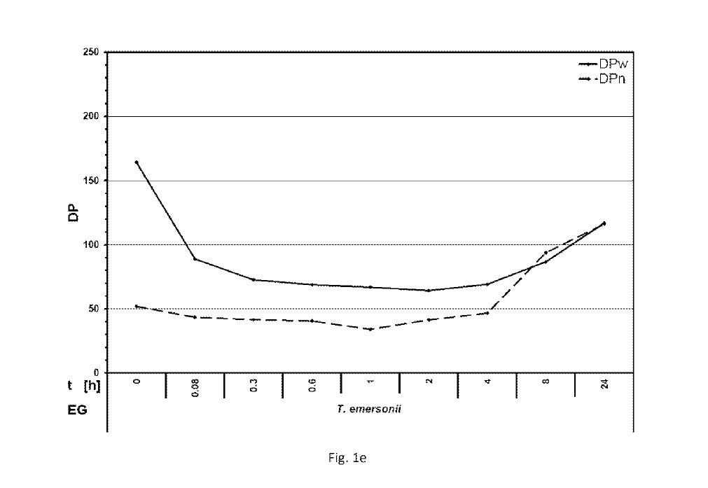

The experimental results are shown in the appended FIGS. 1a to 1e in the form of graphs.

The starting value of the DP_{W} of the sample before the cellulase is added (0-h sample) is between 160 and 220, with a mean of 190.

All the percentages stated hereinbelow refer to the starting mean DP_{W} of 190.

During the hydrolysis experiments with endoglucanases from A. niger, T. maritima, T. emersonii and T. longibrachiatum, the DP_{W} drops by 37% to 53% during the first 5 min and is therefore between 90 and 120. After 24 h, the DP_{W} in the hydrolysis experiments with the A. niger, T. maritima and T. longibrachiatum endoglucanases drops by a further 10% to 21% to 50% to 37% of the starting value.

The DP_{W} of the hydrolysis with the B. amyloliquefaciens endoglucanase after 20 min is 40% of the starting value. After hydrolysis for 24 h, the DP_{W} is lowered by a further 5% to 35% of the starting value.

The DP_{W} of the hydrolysis of T. emersonii endoglucanase is lowered by 52% of the starting value after hydrolysis for 20 min. After hydrolysis for 2 h, the DP_{W} has attained a value of 70, which is 36% of the starting value. Thereafter, the DP_{W} climbs to 120 until the end of the reaction time of 24 h. In the hydrolysis of Sigmacell with T. emersonii endoglucanase, too, both the DP_{W} and the DP_{N} climb after hydrolysis for 3 h. It can therefore not be assumed that the climb observed is an error of measurement. A possible cause would be an increased degradation of short chains, whereby the existing longer chains would be emphasized more in comparison.

When using the endoglucanases from B. amyloliquefaciens and T. maritima, no further degradation of Avicel can be observed after hydrolysis for 2 h. In the hydrolysis experiments with the endoglucanases from A. niger and T. emersonii, a further reduction of the DP_{W} can be observed until the end of the experiment.

When using all endoglucanases, the DP_{W} is reduced the most relative to the starting value during the first 5 min of the experiment in comparison with the remainder of the experimental period. The DP_{W} drops to approximately 50% during the first 1 to 2 h. In comparison with the other endoglucanases, the lowest DP_{W}, with a value of 65, can be achieved when using the B. amyloliquefaciens endoglucanase.

The polydispersity, which is listed in the table hereinbelow, was calculated from the DP_{W} and the DP_{N}:

| | Degree of polymerization | | |
|---|---|---|---|
| Endoglucanase | DP_{W} | DP_{N} | Polydispersity |
| A. niger | 75 [symbol] | 40 [symbol] | 1.8 |
| B. amyloliquefaciens | 65 [symbol] | 40 [symbol] | 1.6 |
| T. maritima | 95 [symbol] | 45 [symbol] | 2.1 |
| T. longibrachiatum | 65 [symbol] | 40 [symbol] | 1.6 |
| T. emersonii (2 h) | 75 [symbol] | 40 [symbol] | 1.9 |

Determination of DP_{W}, DP_{N} and polydispersity (PD) by GPCo (software: ASTRA)

Arrows indicate the respective trend

The polydispersity decreases with decreasing DP_{W}. Accordingly, there is a correlation between polydispersity and the degree of degradation of the cellulose.

### Example 4

### Mass Balance of the Hydrolysis of Avicel

The hydrolysis of cellulose generates not only the desired insoluble cellooligomers, but also soluble cellooligomers and glucose. This example deals with the quantification and assessment of the loss of the cellulose employed as a result of the production of soluble cellooligomers and glucose. The dissolved cellooligomers and glucose were quantified with the aid of an HPLC and the PAHBAH test. The aqueous supernatant of the Avicel hydrolysis samples was used for these studies. The glucose and cellobiose concentrations were determined by HPLC analysis. The concentration of these two dissolved sugars increases with time for all the endoglucanases used.

The mass distribution of Avicel after the enzymatic hydrolysis was compiled in the table which follows:

| | | Soluble | | | |
|---|---|---|---|---|---|
| | Insol- | | | Unknown | |
| | uble | Re- | Glucose | soluble | Mass |
| | Cellu- | ducing | and | cellooligomers | bal- |
| | lose | sugars | cellobiose | (Glc₃-Glc₆) | ance |
| Endoglucanase | <[%] | >[%] | [%] | <[%] | [%] |
| A. niger | 81.0 | 10.4 | 10.9 | 0(−0.5) | 91.4 |
| B. amyloliquefaciens | 75.0 | 31.7 | 25.6 | 6.1 | 106.7 |
| T. maritima | 96.6 | 7.1 | 6.7 | 0.4 | 103.7 |
| T. longibrachiatum | 56.0 | 34.8 | 32.8 | 2.0 | 90.8 |
| T. emersonii | 29.0 | 61.8 | 50.8 | 11.0 | 100.8 |

Insoluble cellooligomers measured by determination of the cellulose loss after 24 h;
reducing sugars measured by PAHBAH test after 24 h;
glucose and cellobiose measured by HPLC after 24 h;
unknown soluble cellooligomers (difference between PAHBAH test and HPLC analysis) after 24 h;
percentage based on the formed glucose and cellobiose upon complete conversion of the cellulose employed

At 11% unknown soluble cellooligomers, the T. emersonii endoglucanase is the highest producer of unknown soluble cellooligomers. The B. amyloliquefaciens endoglucanase produces 6.1% unknown soluble cellooligomers. When using the T. longibrachiatum endoglucanase, 2% of unknown soluble cellooligomers are generated. At 0.5% and 0%, the use of the T. maritima and A. niger endoglucanases results in a very low amount of unknown soluble cellooligomers or amounts which cannot be measured at all. Since the T. emersonii and B. amyloliquefaciens endoglucanases produce the highest amount of soluble cellooligomers in comparison with the other endoglucanases, these two endoglucanases might be used for the production of soluble cellooligomers.

At 29% cellulose after hydrolysis, the use of the T. emersonii endoglucanase mainly gives rise to glucose, cellobiose (51%) and soluble cellooligomers (11%). When using the B. amyloliquefaciens endoglucanase at 75%, the A. niger endoglucanase at 81% and the T. maritima endoglucanase at 96.6% insoluble cellulose after the hydrolysis, the best cellulose yields are achieved in comparison with the other endoglucanases. Therefore, the B. amyloliquefaciens and A. niger endoglucanases are of particular interest for an industrial process with the aim of producing insoluble cellooligomers when the insoluble cellulose takes the form of relatively short-chain cellooligomers.

### Example 5

### Enzymatic Hydrolysis of α-Cellulose by Means of Endoglucanases from A. niger, B. amyloliquefaciens, T. maritima, T. longibrachiatum and T. emersonii

The chain length distribution of the hydrolysis samples of α-cellulose was determined with the aid of GPOₒ.

A. niger

Composition of the hydrolysis mixture:

10 mg/ml alpha-cellulose,

10 U/ml A. niger endoglucanase,

3 U/ml A. niger β-glucosidase,

40° C., 0.1 M acetate buffer pH 4.5

α-Cellulose which had not been treated with enzyme shows the following characteristic course: The cellulose chain concentration increases in the range 20 to 90 glucose units. The slope goes down in the range 90 to 200 glucose units and then climbs again in the range 200 to 450 glucose units.

After hydrolysis for 5 min with the A. niger endoglucanase, the shoulder is no longer visible. The upper end of the chain length distribution is shifted towards shorter chain lengths in comparison with the course of the non-hydrolyzed sample. Likewise, the maximum of the distribution is shifted towards shorter chains. The distribution is between 10 and 800 glucose units, with a maximum of 120. Until 19 h, the chain length distribution continues to narrow. The curve is now in the range between 10 and 450 glucose units, with a maximum at 75. Therefore, a reduction of the maximum by 80% and of the upper end of the chain length distribution by 75% is achieved.

B. amyloliquefaciens

Composition of the hydrolysis mixture:

10 mg/ml alpha-cellulose,

10 U/ml B. amyloliquefaciens endoglucanase,

3 U/ml Agrobacterium sp. β-glucosidase,

40° C., 0.1 M phosphate buffer pH 6

The chain length distribution of the hydrolysis of α-cellulose by B. amyloliquefaciens endoglucanase was determined by GPOₒ. After hydrolysis for 5 min with the A. niger endoglucanase, only a shallowing of the decline between 10 and 30 glucose units of the chain length distribution can be discerned instead of the shoulder. After a reaction time of 20 min, this shallowing is no longer discernible; instead, a near-Gaussian chain length distribution can be observed. At the end of the experiment, the distribution of the chain lengths is between 10 and 400 glucose units with a maximum at 80 glucose units. The enzymatic hydrolysis reduces the maximum by 80% and the upper end by 75%.

T. maritima

Composition of the hydrolysis mixture:

10 mg/ml alpha-cellulose,

10 U/ml T. maritima endoglucanase,

3 U/ml A. niger β-glucosidase,

40° C., 0.1 M acetate buffer pH 4.5

The chain length distribution of the hydrolysis of α-cellulose by T. maritima endoglucanase was determined by GPOₒ. The 5-min sample from the hydrolysis of α-cellulose by means of T. maritima endoglucanase and analyzed by GPCₒ demonstrates a chain length distribution of between 10 and 1800 glucose units. In comparison with the sample without enzyme, cellooligomers are present in the range 10 to 20 glucose units. After 5 min, the maximum has shifted from 450 to 350. A shoulder is no longer pronounced, but a slight shallowing of the incline can be discerned at approximately 120 glucose units. The incline of the chain length distribution in the range between 10 and 30 glucose units is shallower in comparison with the incline in the range 30 glucose units to the maximum. The upper end of the chain length distribution and the maxima of the curves shift towards shorter chain lengths during hydrolysis. After a reaction time of 19 h, the curve is between 10 and 600 and the maximum is at 150 glucose units. Thus, the maximum of the chain length distribution is reduced by 65% and the upper end of the chain length distribution by 65%.

T. longibrachiatum

Composition of the hydrolysis mixture:

10 mg/ml alpha-cellulose,

10 U/ml T. longibrachiatum endoglucanase,

3 U/ml A. niger β-glucosidase,

40° C., 0.1 M acetate buffer pH 4.5

The chain length distribution of the hydrolysis of α-cellulose by T. longibrachiatum endoglucanase was determined by GPOₒ. The upper end of the chain length distribution of the hydrolysis of α-cellulose by T. longibrachiatum endoglucanase shifts towards shorter chain lengths after a hydrolysis time of 5 min. After this reaction time, the chain length distribution is between 10 and 1500 glucose units. In comparison with the sample without enzyme, the maximum has shifted from 450 to 200 glucose units. Likewise, the shoulder has shifted towards shorter chain lengths and is at approximately 25 glucose units. During a reaction time of 19 h, the upper end of the chain length distribution continues to shift towards shorter chain lengths. Likewise, the maxima of the curves shift towards shorter chains. Since the chain length distribution narrows in the course of the experiment, the height of the maxima during the experiment increases. After 19 h, the chain length distribution is between 10 and 400 and the maximum at 75 glucose units.

Within the hydrolysis time of 19 h, the maximum is reduced by 80% and the upper end of the chain length distribution by 75%.

T. emersonii

Composition of the hydrolysis mixture:

10 mg/ml alpha-cellulose,

10 U/ml T. emersonii endoglucanase,

3 U/ml A. niger β-glucosidase,

40° C., 0.1 M acetate buffer pH 4.5

The chain length distribution of the hydrolysis of α-cellulose by T. emersonii endoglucanase was determined by GPOₒ. The chain length distribution of the sample without enzyme is between 20 and 1800 glucose units with a maximum at 450 glucose units. The chain length distribution has a shallower region (shoulder) between 90 and 200 glucose units. The upper end of the chain length distribution of the hydrolysis of α-cellulose by the T. emersonii endoglucanase shifts towards shorter chain lengths after hydrolysis for 5 min. After this reaction time, the chain length distribution is between 10 and 1500 glucose units. In comparison with the sample without enzyme, the maximum has shifted from 450 to 280 glucose units. Likewise, the shoulder has shifted towards shorter chain lengths and is at approximately 30 glucose units. During a reaction time of 19 h, the upper end of the chain length distribution continues to shift towards shorter chain lengths. Likewise, the maxima of the curves shift towards shorter chains. Since the chain length distribution narrows in the course of the experiment, the height of the maxima during the experiment increases. After 19 h, the chain length distribution is between 10 and 300 and the maximum at 70 glucose units. The enzymatic hydrolysis reduces the maximum by 85% and the upper end of the chain length distribution by 80%.

### Summary

Upon comparison of the presented endoglucanases, the endoglucanases from A. niger and B. amyloliquefaciens demonstrate the most rapid cellulose degradation. The endoglucanases from T. longibrachiatum and T. emersonii degrade α-cellulose more slowly. However, the chain length distributions after 19 h are within the same range. When using the endoglucanase from T. maritima, a-cellulose is likewise degraded more slowly in comparison with the hydrolysis by endoglucanases from A. niger and B. amyloliquefaciens. After a reaction time of 19 h, however, α-cellulose is not degraded to the same degree as when using the other endoglucanases. It can be observed in particular for the slower endoglucanases that the maximum first shifts towards shorter chains and cellooligomers having a chain length of 10 to 30 glucose units are formed. As the reaction time progresses, the chain length distribution and its maxima continue to shift towards shorter chain lengths. The chain length distribution is thereby narrowed and the height of the maximum increases.

### Example 6

### Determination of the Degree of Polymerization DP of Enzymatically Hydrolyzed Alpha-Cellulose

GPC analyses were carried out to determine the DP of enzymatically hydrolyzed alpha-cellulose (of example 5).

### Sample Composition GPCo:

2 mg/ml cellulose lyophilizate in DMF/19% EMIM Ac (v/v) Mobile phase: DMF/10%, EMIM Ac (v/v), 50° C.

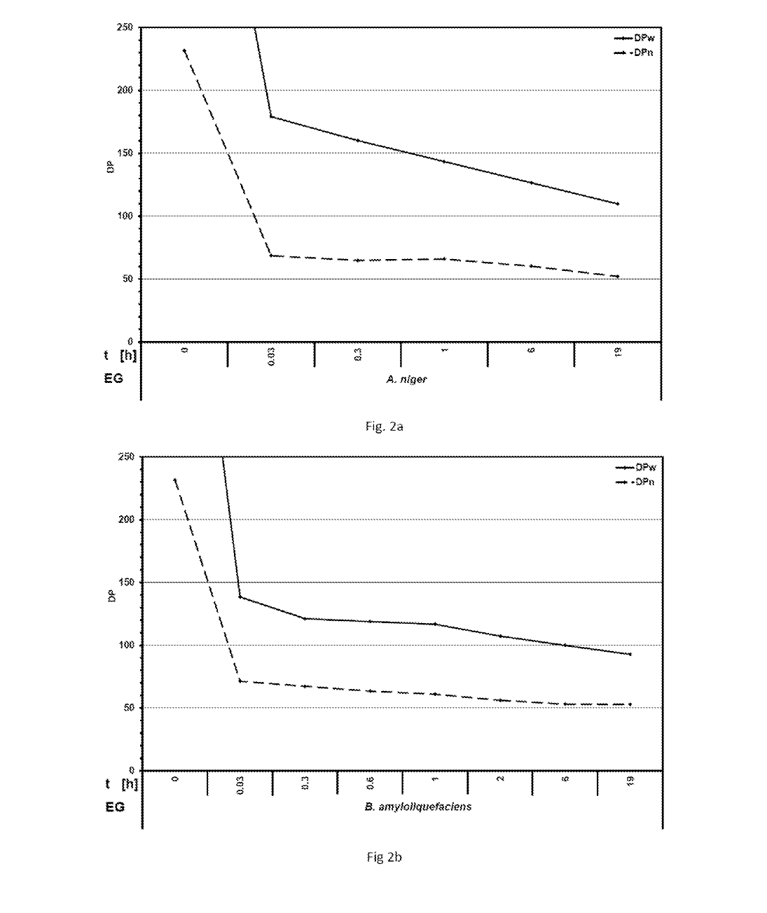

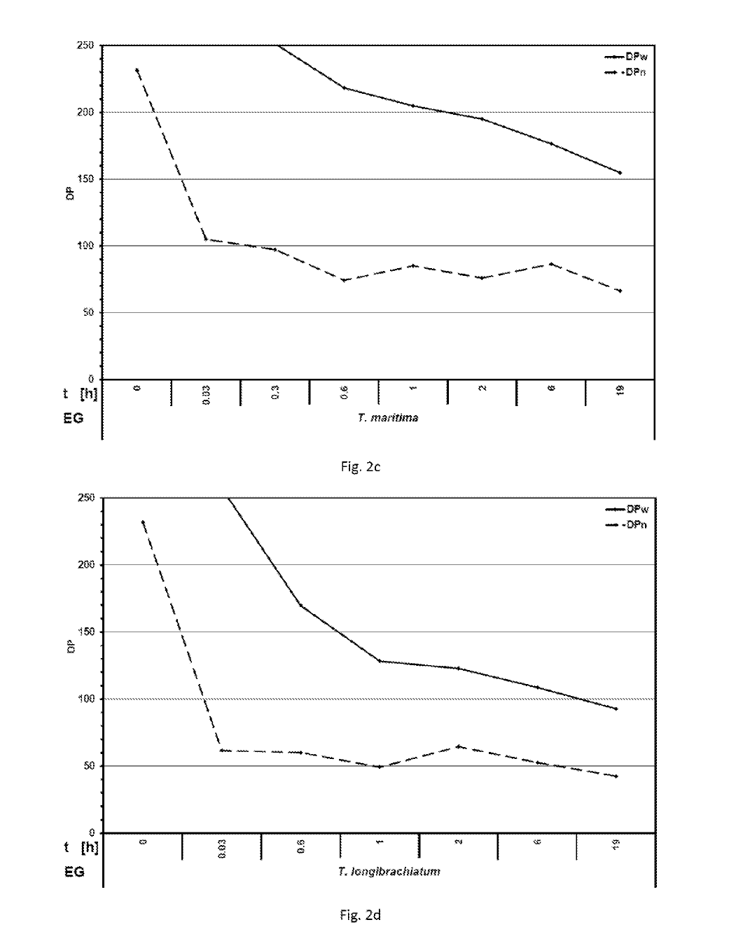

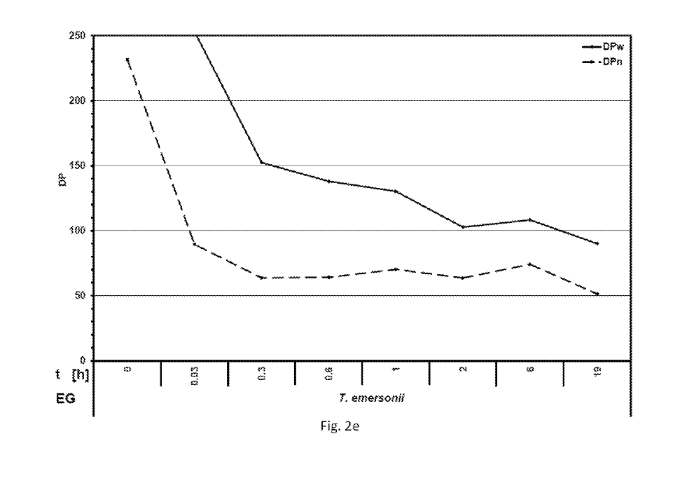

The experimental results are shown in the appended FIGS. 2a to 2e

No enzyme solution was added to the 0-h samples, and the cellulose lyophilizate was analyzed by GPCo. An α-cellulose sample which had previously been incubated in acetate buffer and subsequently lyophilized was used as reference. The DP_{W} of the unhydrolyzed α-cellulose samples amounts to 550. The DP_{N} amounts to 230.

After hydrolysis for 5 min, the DP_{W} of the A. niger samples drops to 180. A reduction of the DP_{W} by at least 50% is thus achieved. The DP_{W} continues to drop continuously and, after 19 h, attains a value of 110 and thus approx. 25% of the initial value. This demonstrates that the reduction of the DP_{W} slows down over time. After a reaction time of 5 min, the DP_{N} drops from originally 240 to 140. The DP_{N} drops to 50 when the experiment ends after 19 h.

The DP_{W} of the B. amyloliquefaciens sample drops to 140 within 5 min. After this reaction time, the DP_{N} amounts to 70. After 19 h, the DP_{W} amounts to 90 and the DP_{N} to 50.

The DP_{W} of the sample when using the T. emersonii endoglucanase after hydrolysis for 20 min is 150. When using the T. longibrachiatum endoglucanase, a DP_{W} of 170 is attained after 40 min. Employing the endoglucanases from A. niger and B. amyloliquefaciens, values in this region are already attained after 5 min. Therefore, the reduction of the DP_{W} when using the T. emersonii and T. longibrachiatum endoglucanases proceeds more slowly than when using the A. niger and B. amyloliquefaciens endoglucanases. After hydrolysis for 19 h with the T. emersonii endoglucanase, a DP_{W} of 90 and a DP_{N} of 50 are attained. When using the T. longibrachiatum endoglucanase, a DP_{W} of 90 and a DP_{N} of 40 are attained after hydrolysis for 19 h. In the hydrolyses of Avicel and Sigmacell, the DP_{W} and the DP_{N} increase after approximately 1 h hydrolysis with the T. emersonii endoglucanase. The increase of the DP cannot be observed when using α-cellulose. The increase of the DP when using Avicel and Sigmacell at a DP_{W} of approximately 70 a, this DP_{W} is not attained when using α-cellulose. Accordingly, an increase would be possible which is dependent on the DP_{W}. When using the T. maritima endoglucanase, too, a degradation of α-cellulose can be observed. After hydrolysis for 19 h, however, the DP_{W} has a value of 150 and is therefore higher than when using the other endoglucanases. With a DP_{N} value of 65 after hydrolysis for 19 h, the DP_{N}, too, is higher than in comparison with the other endoglucanases.

Upon comparison of the hydrolysis of α-cellulose with the five endoglucanases used, the lowest DP_{W} are attained when using the endoglucanase from B. amyloliquefaciens, T longibrachiatum and T. emersonii with a reaction time of 19 h. These values are between 90 and 93.

A reduction of the DP_{W} until the end of the experiment can be observed for all endoglucanases. As for Avicel, the polydispersity was calculated from the DP_{W} and the DP_{N} (cf. table hereinbelow)

| | Degree of polymerization | | |
|---|---|---|---|
| Endoglucanase | D_{W} | DP_{N} | Polydispersity |
| A. niger | 110 [symbol] | 50 [symbol] | 2.2 |
| B. amyloliquefaciens | 90 [symbol] | 50 [symbol] | 1.8 |
| T. maritima | 155 [symbol] | 65 [symbol] | 2.3 |
| T. longibrachiatum | 95 [symbol] | 45 [symbol] | 2.1 |
| T. emersonii | 90 [symbol] | 50 [symbol] | 1.8 |

DP_{W}, DP_{N} and CLD determination by GPCo (software: ASTRA) arrows indicate the respective trend

When using α-cellulose, too, a correlation exists between DP_{W} and polydispersity.

### Example 7

### Mass Balance of the Hydrolysis of Alpha-Cellulose

The results are compiled in the table hereinbelow:

| | | Soluble | | | |
|---|---|---|---|---|---|
| | Insol- | | | Unknown | |
| | uble | Re- | Glucose | soluble | Mass |
| | Cellu- | ducing | and | cellooligomers | bal- |
| | lose | sugars | cellobiose | (Glc₃-Glc₆) | ance |
| Endoglucanase | <[%] | >[%] | [%] | >[%] | [%] |
| A. niger | 94.5 | 9.5 | 6.8 | 2.7 | 104.0 |
| B. amyloliquefaciens | 64.5 | 14.2 | 14.9 | 0.7 | 79.4 |
| T. maritima | 87.1 | 4.8 | 5.0 | 0 (−0.2) | 91.9 |
| T. longibrachiatum | 64.5 | 19.9 | 16.8 | 3.1 | 84.4 |
| T. emersonii | 45.7 | 46.8 | 19.4 | 27.4 | 92.5 |

Insoluble cellooligomers measured by determination of the cellulose loss after 19 h;
reducing sugars measured by PAHBAH test after 19 h;
glucose and cellobiose measured by HPLC after 19 h;
unknown soluble cellooligomers (difference between PAHBAH test and HPLC analysis) after 19 h;
percentage based on the formed glucose and cellobiose upon complete conversion of the cellulose employed

Using the endoglucanases from T. maritima and A. niger, 87.1% and 94.5% insoluble cellulose were measured after the hydrolysis. Therefore, these two enzymes are, owing to the small loss of α-cellulose, of particular interest for an industrial process in which soluble cellooligomers and glucose are not the aim of the production when the insoluble cellulose takes the form of short-chain cellooligomers. The DP_{W} of 150 after 19 h by means of T. maritima endoglucanase is approximately 50 units above the DP_{W} of the other endoglucanases after the same hydrolysis time. Thus, while few by-products are formed with this endoglucanase, the DP_{W} is not reduced to the same extent as when the other endoglucanases are used. With a DP_{W} of 110, the use of the A. niger endoglucanase results in a DP_{W} in the same range as the use of the endoglucanases from B. amyloliquefaciens, T emersonii and T. longibrachiatum. The reduction of the DP_{W} in the same ratio, and the low production of glucose and soluble cellooligomers, in comparison with the other endoglucanases makes the A. niger endoglucanase the favorite endoglucanase for the enzymatic hydrolysis of α-cellulose.

With 0.7% and 0%, the endoglucanases from B. amyloliquefaciens and T. maritima produce virtually no soluble cellooligomers, or none at all. The endoglucanases from A. niger and T. longibrachiatum produce 2.7% and 3.1%, respectively, of unknown cellooligomers. In comparison with the other endoglucanases, the T. emersonii endoglucanase produces the largest amount of unknown soluble cellooligomers with 27.4%. Thus, the T. emersonii endoglucanase is well suited for producing soluble cellooligomers which are larger than cellobiose.

### Example 8

### Enzymatic Hydrolysis of Sigmacell by Means of Endoglucanases from a niger, B. amyloliquefaciens, T. maritima, T. longibrachiatum and T. emersonii

The chain length distribution of the hydrolysis samples of α-cellulose and of Sigmacell was determined with the aid of GPOₒ.

A. niger

Composition of the hydrolysis mixture:

10 mg/ml Sigmacell,

10 U/ml A. niger endoglucanase,

3 U/ml A. niger β-glucosidase,

40° C., 0.1 M acetate buffer pH 4.5

The chain length distribution of Sigmacell without enzymatic degradation is in the range between 10 and 1000 glucose units, with a maximum at 300-400 glucose units, and has a shallower incline in the range between 10 and 50 glucose units than in the range between 50 glucose units and the maximum. After hydrolysis with the A. niger endoglucanase for 5 minutes, the shallow segment is between 10 and 20 glucose units. The chain length distribution is between 10 and 500 glucose units, with a maximum at 100 glucose units. After hydrolysis for 6 h, the upper end of the chain length distribution is between 10 and 350 glucose units and is therefore shifted towards shorter chain lengths by 150 glucose units in comparison with the chain length distribution of the 5-min sample. No shallowing in the left-hand section of the curve can be discerned in the 6-h hydrolysis sample.

B. amyloliquefaciens

Composition of the hydrolysis mixture:

10 mg/ml Sigmacell,

10 U/ml B. amyloliquefaciens endoglucanase,

3 U/ml A. niger β-glucosidase,

40° C., 0.1 M acetate buffer pH 4.5

The chain length distribution of the 5-min sample of the hydrolysis of Sigmacell by endoglucanase from B. amyloliquefaciens is between 10 and 400 glucose units. After this reaction time, no shallowing can be observed between 15 and 50 glucose units. Until the end of the experiment after 20 h, the upper end of the chain length distribution continues to shift continuously towards shorter chains. The distribution after this reaction time is between 10 and 300 glucose units. The maximum likewise shifts towards shorter chain lengths and is at 65 glucose units.

T. maritima

Composition of the hydrolysis mixture:

10 mg/ml Sigmacell,

10 U/ml T. maritima endoglucanase,

3 U/ml A. niger β-glucosidase,

40° C., 0.1 M acetate buffer pH 4.5

After hydrolysis for 5 min with the endoglucanase from T. maritima, the chain length distribution is in the same range as untreated Sigmacell. The maximum of the chain length distribution is shifted towards shorter chain lengths by 100 glucose units, and an increase in short cellulose chains can be discerned in the range from 10 to 200 glucose units. Until the end of the experiment with a reaction time of 20 h, the chain length distribution continues to shift towards shorter chain lengths; at this point in time, it is between 10 and 400 glucose units. Using the T. maritima endoglucanase in comparison with the hydrolysis experiments on Sigmacell using A. niger and B. amyloliquefaciens endoglucanases, a slower degradation of Sigmacell is observed.

T. longibrachiatum

### Composition of the Hydrolysis Mixture:

10 mg/ml Sigmacell,

10 U/ml T. longibrachiatum endoglucanase,

3 U/ml Agrobacterium sp. β-glucosidase,

40° C., 0.1 M acetate buffer pH 4.5

After hydrolysis of Sigmacell for 5 min using endoglucanase from T. longibrachiatum, the chain length distribution is between 15 and 800 glucose units. The shallowing in the front range of the chain length distribution is still discernible at this point in time between 15 and 60 glucose units, but within a shorter range than in the case of untreated Sigmacell. The upper end of the chain length distribution and the maximum of the chain length distribution continues to shift towards shorter chains during the experiment, and the shallowing continues to decrease during the experiment until it is no longer discernible. After 20 h, the curve is between 10 and 300 glucose units, with a maximum at 80 glucose units.

T. emersonii

Composition of the hydrolysis mixture:

10 mg/ml Sigmacell,

10 U/ml T. emersonii endoglucanase,

3 U/ml A. niger β-glucosidase,

40° C., 0.1 M acetate buffer pH 4.5

The chain length distribution of the 20-h sample shows an unexpected course in comparison with the other hydrolysis samples. After Sigmacell has been hydrolyzed for 1 h by means of endoglucanase from T. emersonii, the chain length distribution is in the range from 10 to 300 glucose units, with a maximum of 70 glucose units. Thereafter, an increase in the longer chains can be observed, with the chain length distribution range continuing to narrow. As a result of the shift of the chain length distribution towards longer chain lengths, the maximum of the curve likewise shifts towards longer chain lengths. After a hydrolysis time of 6 h, the chain length distribution is between 10 and 250 glucose units, with a maximum at 100 glucose units.

### Summary:

Upon comparison of the degradation of Sigmacell by means of the five endoglucanases employed, a rapid degradation is achieved using the A. niger and the B. amyloliquefaciens endoglucanases. The degradation rates of the T. maritima and the T. longibrachiatum endoglucanases are slower. When using the endoglucanases from A. niger, B. amyloliquefaciens, T. maritima and T. longibrachiatum, the upper end of the chain length distribution after hydrolysis of Sigmacell for 19 h is at 300 to 400 glucose units. Using the T. emersonii endoglucanase, an increase of longer chains can be observed after a hydrolysis time of 1 h. When using Avicel and the T. emersonii endoglucanase, an increase of longer cellulose chains was likewise observed after a hydrolysis time of 2 h, which is why it cannot be assumed that this observation is an error of measurement. The cause for this observation is unclear. A possibility would be increased degradation of short chains, whereby the existing longer chains would be emphasized more in comparison.

### Example 9

### Determination of the Degree of Polymerization DP of Enzymatically Hydrolyzed Sigmacell

GPC analyses were carried out to determine the DP of enzymatically hydrolyzed Sigmacell (of example 8).

### Sample Composition GPCo:

2 mg/ml cellulose lyophilizate in DMF/19% EMIM Ac (v/v)

Mobile phase: DMF/10%, EMIM Ac (v/v), 50° C.

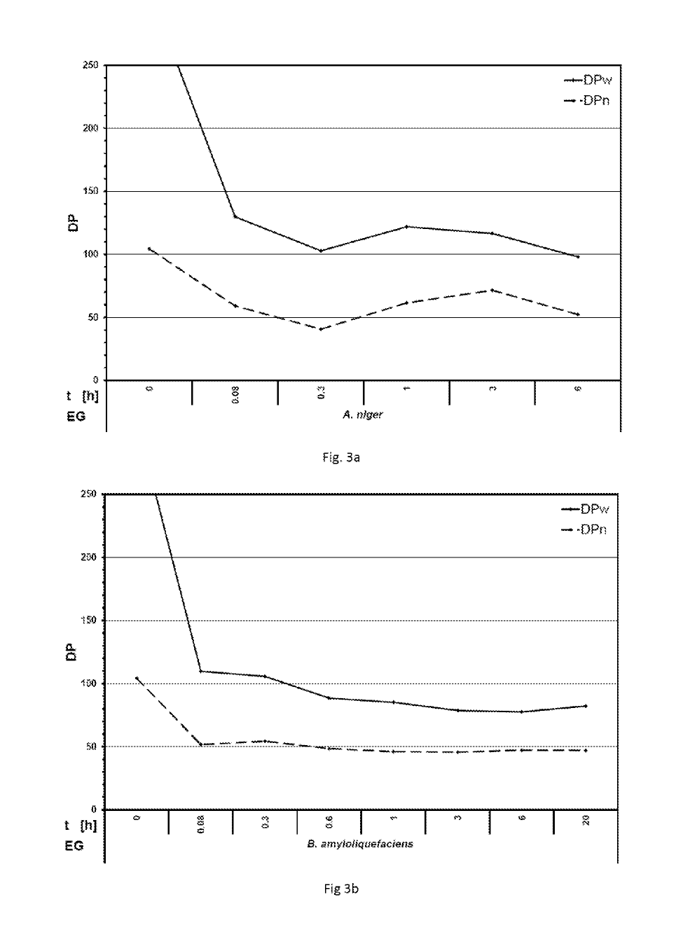

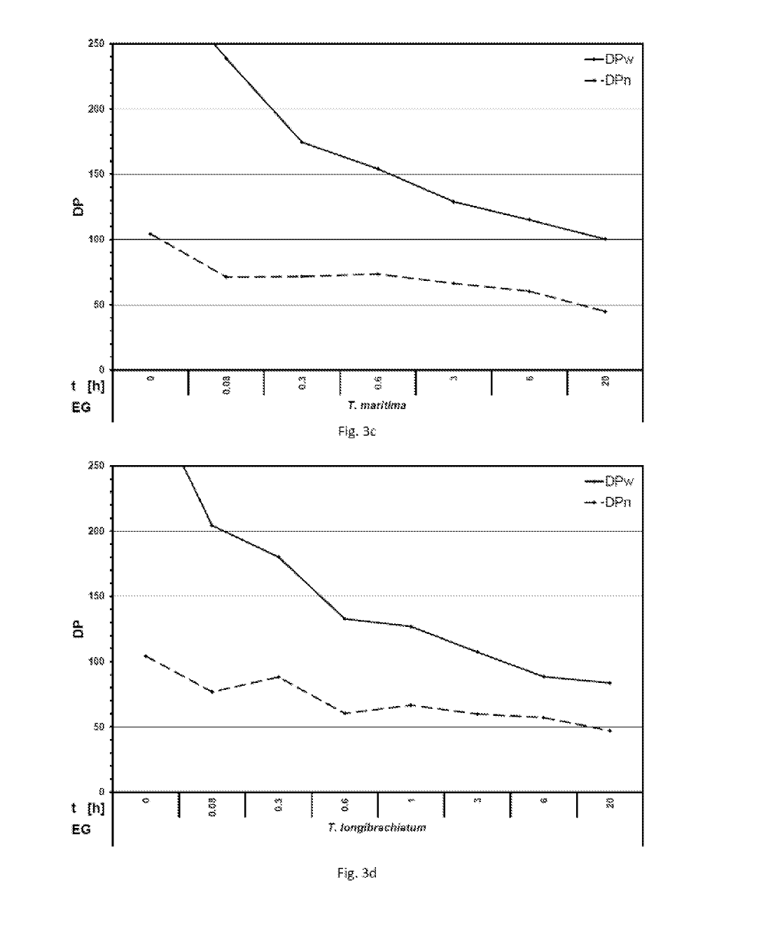

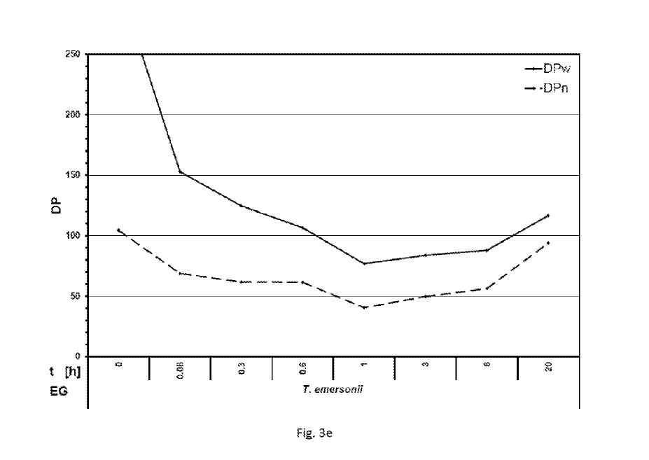

The experimental results are shown in the appended FIGS. 3a to 3e.

After hydrolysis for 5 min using A. niger endoglucanase, the DP_{W} drops to 130. The DP_{N} drops from 110 to 60. Therefore, both the DP_{W} and the DP_{N} are reduced by at least 55%. After a hydrolysis time of 20 min, a DP_{W} of 100 and a DP_{N} of 40 are attained. Therefore, hydrolysis proceeds more slowly. With a DP_{W} of 120, the DP_{W} of the 1-h and 3-h samples increases slightly. The DP_{N} shows an analogous course. The DP_{W} of 100 and the DP_{N} of 50 after a reaction time of 6 h support the assumption that the increase of the DP_{W} and the DP_{N} after a hydrolysis time of 1 h and 3 h is probably likewise based on disturbances to the measurement.

When using the B. amyloliquefaciens endoglucanase, it is possible to reduce the DP_{W} to 50% of the initial value after a hydrolysis time of 5 min. Until the end of the experiment, the DP_{W} continues to drop to 80 and then stagnates. An analogous course can be observed for the DP_{N}. This value is likewise halved after a hydrolysis time of 5 min and then stagnates after 40 min at a DP_{N} of 50. A termination of the degradation is achieved. The hydrolysis of Sigmacell by means of endoglucanase from T. emersonii and T. longibrachiatum proceeds more slowly than the hydrolysis by the endoglucanases from A. niger and B. amyloliquefaciens. After a reaction time of 5 min, the DP_{W} when using T. emersonii endoglucanase is 150. After a reaction time of 1 h, the DP_{W} continues to drop to 75. At this point in time, the DP_{N} is at 40. After hydrolysis for 3 h, an increase of the DP_{W} can be observed. With the hydrolysis of α-cellulose by the T. emersonii endoglucanase, too, both the DP_{W} and the DP_{N} increases after hydrolysis for 2 h. A possible cause would be an increased degradation of short chains, whereby the existing longer chains would be emphasized more in comparison.

When using the T. longibrachiatum endoglucanase, a DP_{W} of 200 is attained after a reaction time of 5 min. After a reaction time of 1 h, the DP_{W} continues to drop to 130. At this point in time, the DP_{N} is 65. After 1 h, the cellulose continues to be degraded when using the T. longibrachiatum endoglucanase, and, after the termination of the experiment, a DP_{W} of 80 and a DP_{N} of 50 is attained.

The hydrolysis of Sigmacell using the T. maritima endoglucanase proceeds more slowly than the hydrolyses of Sigmacell by the endoglucanases from A. niger, B. amyloliquefaciens, T. emersonii and T. longibrachiatum. After a reaction time of 20 h, a DP_{W} of 100 and a DP_{N} of 45 are in the same range as when using the A. niger endoglucanase after a reaction time of 6 h.

After hydrolysis for 20 h using the endoglucanases presented, the DP_{W} can be lowered to less than half of the initial value. The endoglucanases used differ in respect of their reaction rate and the DP_{W} which is attained at the end of the experiment. The lowest DP values can be attained using the endoglucanase from B. amyloliquefaciens and T. longibrachiatum. With the endoglucanase from B. amyloliquefaciens, the DP_{W} stagnates at 80. Accordingly, a termination of the degradation is achieved. With the endoglucanases from A. niger, T maritima and T. emersonii, the DP_{W} drops until the end of the experiment. Therefore, no termination of degradation is achieved.

Owing to the fact that the reaction rate of the endoglucanases from A. niger and B. amyloliquefaciens is more rapid in comparison with the other endoglucanases, these endoglucanases are of particular interest for further experimental studies.

As has been the case with Avicel and α-cellulose, the polydispersity for Sigmacell, too, was calculated from the DP_{W} and the DP_{N} and can be seen from the table which follows.

| | Degree of polymerization | | |
|---|---|---|---|
| Endoglucanase | DP_{W} | DP_{N} | Polydispersity |
| A. niger (6 h) | 100 [symbol] | 50 [symbol] | 2 |
| B. amyloliquefaciens | 80 [symbol] | 45 [symbol] | 1.7 |
| T. maritima | 100 [symbol] | 45 [symbol] | 2.2 |
| T. longibrachiatum | 85 [symbol] | 45 [symbol] | 1.9 |
| T. emersonii (1 h) | 80 [symbol] | 40 [symbol] | 2 |

DP_{W}, DP_{N} and CLD determination by GPCo (software: ASTRA) arrows indicate the respective trend

The DP and polydispersity results of the enzymatic hydrolysis using the endoglucanase from A. niger and T. emersonii do not correlate with each other. Due to errors of measurement in the hydrolysis samples of the A. niger endoglucanase and an unusual DP rise of the hydrolysis samples of the T. emersonii endoglucanase, however, no DP results after 1 d were used with these endoglucanases. The DP_{W} and the polydispersity of the other endoglucanases with a hydrolysis time of 1 d correlate with each other, as has already been observed for Avicel and α-cellulose.

### Example 10

### Mass Balance of the Hydrolysis of Sigmacell

The readings are compiled in the following table:

| | | Soluble | | | |
|---|---|---|---|---|---|
| | Insol- | | | Unknown | |
| | uble | Re- | Glucose | soluble | Mass |
| | Cellu- | ducing | and | cellooligomers | bal- |
| | lose | sugars | cellobiose | (Glc3-Glc6) | ance |
| Endoglucanase | <[%] | >[%] | [%] | >[%] | [%] |
| A. niger | 87.9 | 12.5 | 9.0 | 3.5 | 100.4 |
| B. amyloliquefaciens | 78 | 20.5 | 18.9 | 1.7 | 98 |
| T. maritima | 100 | 9.1 | 6.9 | 2.2 | 109.1 |
| T. longibrachiatum | 58.2 | 48.6 | 23.2 | 25.4 | 106.8 |
| T. emersonii | 57.0 | 46.2 | 33.5 | 12.7 | 103.2 |

Insoluble cellooligomers measured by determination of the cellulose loss after 20 h;
reducing sugars measured by PAHBAH test after 20 h;
glucose and cellobiose measured by HPLC after 20 h;
unknown soluble cellooligomers (difference between PAHBAH test and HPLC analysis) after 20 h;
percentage based on the formed glucose and cellobiose upon complete conversion of the cellulose employed

Using the endoglucanases from A. niger, 87.9% insoluble cellulose was measured after the hydrolysis. When using the T. maritima endoglucanase, 100% insoluble cellulose was measured. Owing to the small Sigmacell loss, these two enzymes are, therefore, of particular interest for an industrial process in which soluble cellooligomers and glucose are not the aim of the production.

Using the PAHBAH test, a fraction of 9.1% reducing sugars was determined in the hydrolysis of Sigmacell by means of T. maritima endoglucanase. The proportion of 100% insoluble cellulose is, accordingly, too high since at least 9.1% of the cellulose employed has been converted into glucose and soluble cellooligomers. When using the endoglucanases from B. amyloliquefaciens, T. maritima and A. niger, less than 4% unknown soluble cellooligomers are produced. Using the endoglucanases from T. emersonii and T. longibrachiatum, 12.7% and 25.4%, respectively, of unknown soluble cellooligomers are produced. These endoglucanases are therefore suitable for the production of soluble cellooligomers.

### Example 11

### Second Enzymatic Hydrolysis of the Celluloses after Further Pretreatment with Ionic Liquid: (IL Restart)

Using IL restart, the cellulose which has already been degraded by enzymatic hydrolysis for 2 days is redissolved in ionic liquid and subsequently precipitated. The cellulose is degraded once more by the further pretreatment. Using this method, it is possible to study a termination of degradation as caused by the substrate. The results of these studies are described hereinbelow.

The endoglucanases from A. niger, B. amyloliquefaciens and T. maritima were used for these experiments because they are of particular interest for the production of cellooligomers due to the low production of dissolved sugars. These studies were carried out using the short-chain substrate Avicel and the long-chain substrate α-cellulose in order to study if chain length dependency exists.

### Example 11a

### Hydrolysis of Avicel after IL Restart

The samples were analyzed by GPCo to analyze the follow-on experiments on the hydrolysis of Avicel.

### Composition of the Hydrolysis Mixture:

10 mg/ml Avicel,

10 U/ml endoglucanase from A. niger, B. amyloliquefaciens and T. maritima, respectively,

3 U/ml β-glucosidase from A. niger and Agrobacterium sp., respectively,

40° C., 0.1 M phosphate buffer pH 6 and 0.1 M acetate buffer pH 4.5, respectively.

A shift of the upper end of the chain length distribution towards shorter chains in comparison with the simple hydrolysis can be observed for the chain length distributions of the samples for which an IL restart was carried out. The chain length distributions generated by the second hydrolysis differ depending on the endoglucanase employed. When using the A. niger and B. amyloliquefaciens endoglucanase, an increase in cellooligomers with a size of 18 glucose units is discernible after carrying out an IL restart. Owing to the increase in cellooligomers with a size of 18 glucose units, a further maximum at 18 glucose units is observed in the hydrolysis using A. niger endoglucanase after carrying out an IL restart. Using the T. maritima endoglucanase, an increase in cellooligomers with a size of 15 glucose units can be observed after carrying out an IL restart.

### Example 11b

### Determining the Degree of Polymerization DP of Enzymatically Hydrolyzed Avicel after IL Restart

GPC analyses were carried out to determine the DP of enzymatically hydrolyzed Avicel (of example 11a).

### Sample Composition GPCo:

2 mg/ml cellulose lyophilizate in DMF/19% EMIM Ac (v/v)

Mobile phase: DMF/10° A, EMIM Ac (v/v), 50° C.

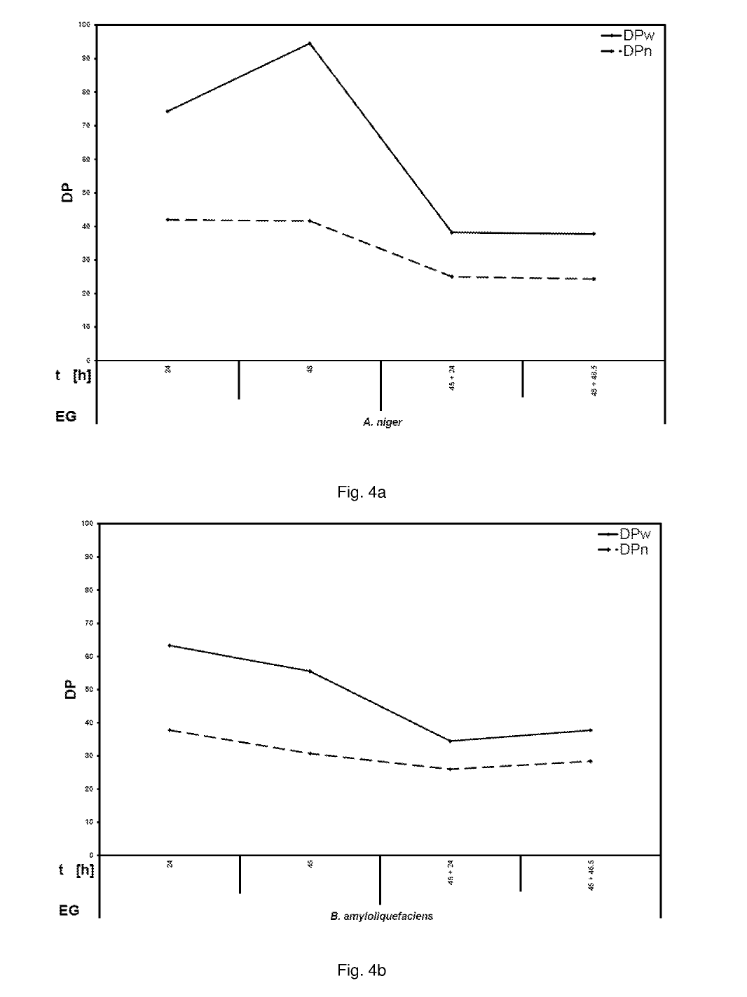

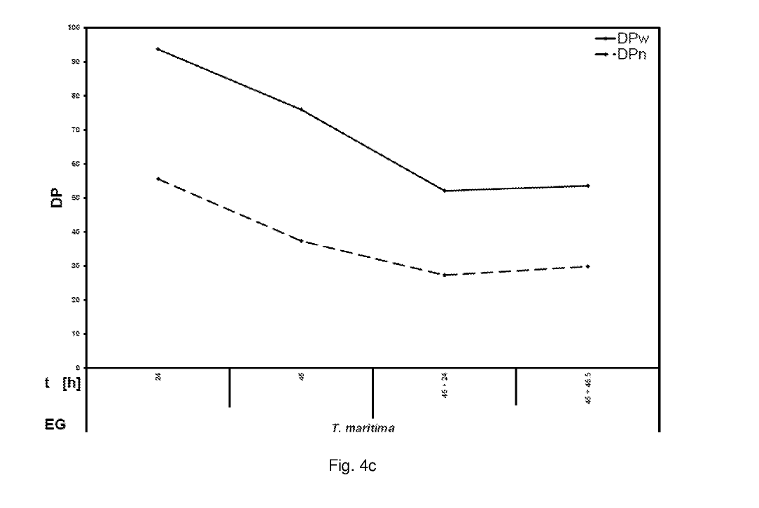

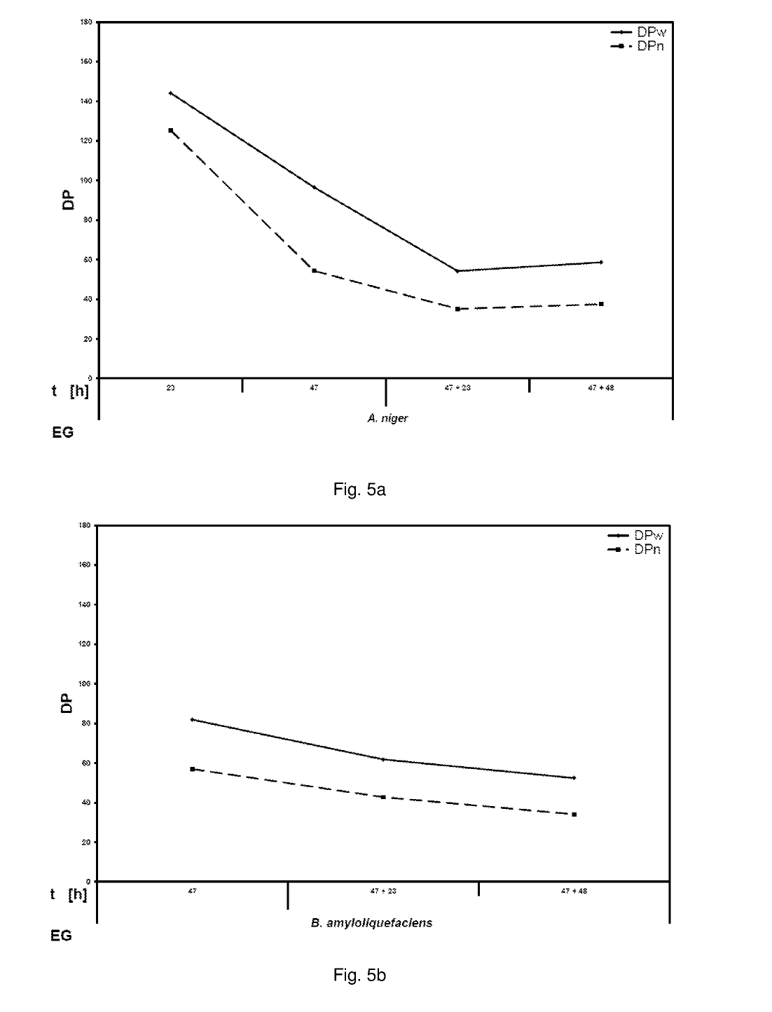

The experimental results are shown in the appended FIGS. 4a to 4c.

After hydrolysis by A. niger endoglucanase for 21 h, a DP_{W} of 85 is achieved. The analysis of the sample which had been incubated for 45 h demonstrates that the cellulose is not degraded any further. Indeed, the DP_{W} climbs to 95. Accordingly, the reaction time of 1 d is sufficient when using this endoglucanase and Avicel.

The treatment by IL restart allows the DP_{W} to be reduced to half of the value after a single hydrolysis. Accordingly, the IL restart method is a suitable method for producing cellulose with a DP_{W} of 40.

When using the B. amyloliquefaciens endoglucanase, the DP_{W} of the samples except for those of the restart experiments is between 55 and 65. When using the IL restart method, a DP_{W} of 35 is obtained.

When using the T. maritima endoglucanase, the DP_{W} of the 1-d and 2-d samples amounts to 80. The IL restart method leads to a DP_{W} of 55. Using this method, the DP_{W} can be reduced by at least 30% compared with that of a simple hydrolysis.

### Example 11c

### Hydrolysis of α-Cellulose after IL Restart

The samples were analyzed by GPC to analyze the follow-on experiments on the hydrolysis of alpha-cellulose.

### Composition of the Hydrolysis Mixture:

10 mg/ml alpha-cellulose,

10 U/ml endoglucanase from A. niger, B. amyloliquefaciens and T. maritima, respectively,

3 U/ml β-glucosidase from A. niger and Agrobacterium sp., respectively,

40° C., 0.1 M phosphate buffer pH 6 and 0.1 M acetate buffer pH 4.5, respectively.

The chain length distributions of the α-cellulose hydrolysis experiments with the enzymes employed show essentially a profile that is analogous to the Avicel hydrolysis. Only the IL restart experiments result in significantly reduced molecular weights. When using the A. niger endoglucanase, an increase of cellooligomers with a size of 18 glucose units can be discerned after carrying out an IL restart. After carrying out an IL restart, the chain length distribution shifts towards shorter chains and is between 10 and 200 glucose units. When using the B. amyloliquefaciens endoglucanase for hydrolyzing α-cellulose, the maximum of the chain length distribution after carrying out an IL restart shifts towards shorter chain lengths by 30 glucose units. After carrying out an IL restart using the T. maritima endoglucanase, no shift of the upper end of the chain length distribution or of the maximum of the chain length distribution towards shorter chain lengths is observed.

### Example 11d

### Determining the Degree of Polymerization DP of Enzymatically Hydrolyzed Alpha-Cellulose after IL Restart

GPC analyses were carried out to determine the DP of enzymatically hydrolyzed alpha-cellulose (of example 11c).

### Sample Composition GPOₒ:

2 mg/ml cellulose lyophilizate in DMF/19% EMIM Ac (v/v)

Mobile phase: DMF/10%, EMIM Ac (v/v), 50° C.

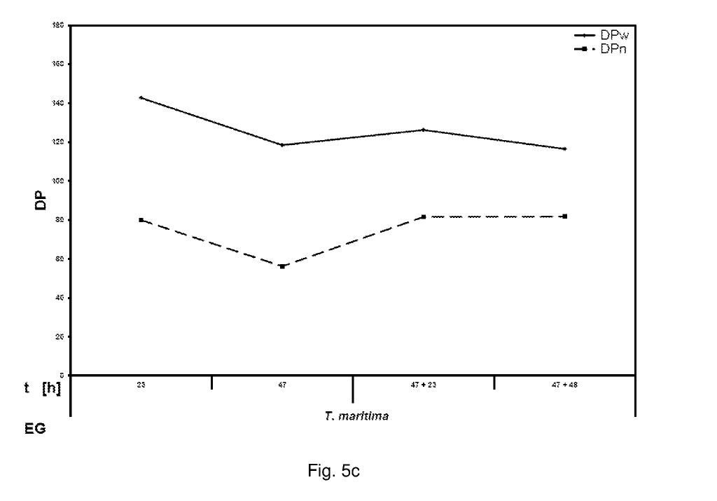

The experimental results are shown in the appended FIGS. 5a to c.

By extending the reaction time to 2 d, a reduction of the DP_{W} can be achieved for the endoglucanases from A. niger and T. maritima. This is unclear for the B. amyloliquefaciens endoglucanase since owing to lyophilization problems the 1 d hydrolysis sample was discarded.

As regards the A. niger endoglucanase, the DP_{W} after 2 d is lowered by 40% when using the IL restart compared to a simple hydrolysis.

When using the IL restart method with the B. amyloliquefaciens endoglucanase, the DP_{W} after 2 d can be lowered by 36% compared with a simple hydrolysis.

Using the T. maritima endoglucanase, a DP_{W} of 140 is attained after hydrolysis for 1 d and a DP_{W} of 120 after hydrolysis for 2 d. By carrying out an IL restart, no further degradation can be achieved in comparison with a simple hydrolysis.

The DP_{W} in a simple hydrolysis drops until the end of the experiment after 2 d; therefore, no termination of degradation is achieved.

Using the IL restart method, α-cellulose with a DP_{W} of 55 can be produced.

The β-glucosidase used in the examples is an optional further configuration of the invention. It has been possible to demonstrate within the scope of studies according to the invention that the use of said enzyme is not mandatory.

It is known that, owing to the presence of cellulose degradation products (in particular cellobiose), β-glucosidase is capable of preventing any product inhibition caused by the endoglucanases. The actual necessity of using this enzyme, however, can be determined by a small number of routine preliminary experiments.

Reference is made to the disclosure of the documents cited herein.

We claim:

1. A process for the production of cellooligomers, where
a) cellulose (cellulosic starting material) is cleaved hydrolytically using at least one endoglucanase (EG) (E.C.3.2.1.4) in an aqueous reaction medium, and
b) the reaction product, which comprises one or more cellooligomers, i.e. a cellooligomer fraction, is isolated from the reaction medium.

2. The process according to claim 1, wherein the cellooligomer(s) formed has (have) a number-average chain length (a number-average degree of polymerization DP_{N}) in the range of from 10 to 100.

3. The process according to any of the preceding claims, wherein the cellooligomer(s) formed has (have) a DP_{N} value in the range of from 15 to 50.

4. The process according to any of the preceding claims, wherein the EG is a natural or recombinantly produced, optionally genetically modified enzyme from microorganisms of the genus Bacillus, Aspergillus or Thermotoga, in particular the species Bacillus amyloliquefaciens, Aspergillus niger or Thermotoga maritima, or a combination of at least two of these natural or recombinant enzymes.

5. The process according to any of the preceding claims, wherein the enzymatic hydrolysis is carried out in an aqueous medium at a pH in the range of approximately 3 to 8, in particular 4 to 7 and/or at a temperature in the range of from 20 to 90° C., in particular from 30 to 80° C. and/or over a duration of 0.1 to 100 hours, in particular 1 to 48 hours.

6. The process according to any of the preceding claims, wherein at least one EG is applied at a concentration of approximately 0.01 to 100, such as, for example, 1 to 50 or 2 to 10 U/ml reaction mixture.

7. The process according to any of the preceding claims, wherein cellulose is employed at a concentration in the range of from 0.1 to 5% (w/v) based on the total volume of the reaction mixture.

8. The process according to any of the preceding claims, wherein the cellulose
a1) is subjected to a pretreatment step by which the crystallinity of the cellulose is reduced, and
a2) the cellulose of step a1) is hydrolyzed enzymatically using the EG.

9. The process according to claim 8, wherein the crystallinity of the cellulose is reduced by treatment in step a1) using ionic liquid, acid and/or mechanical energy input.

10. The process according to claim 9, wherein the ionic liquid is selected among salts which are liquid below a temperature of 100° C. such as, in particular, 1-ethyl-3-methylimidazolium acetate (EMIM Ac) and 3-methyl-N-butylpyridinium chloride ([C4mpy]Cl).

11. The process according to claim 10, wherein, in step a1), cellulose is introduced into the ionic liquid, dissolved therein, optionally under thermal action and subsequently precipitated by adding water, an organic solvent or a mixture thereof, the precipitate is separated off, optionally washed and liquid is optionally removed.

12. The process according to claim 9, wherein, in step a1), the acid treatment is carried out with concentrated phosphoric acid.

13. The process according to claim 9, wherein, in step a1), the mechanical treatment is carried out using a ball mill.

14. The process according to any of the preceding claims, wherein the treatment steps, in particular steps a1) and a2), are repeated once or more than once before the reaction product is isolated.

15. The use of a cellooligomer, produced by a process according to any of the preceding claims, as additive to foodstuffs and feedstuffs, cosmetics or pharmaceuticals, as a detergent additive, as a rheology modifier and as a starting material for organic syntheses.


SEQUENCE LISTING (3 sequences)

SEQ ID NO: 1 - protein, 329 aa, Thermotoga maritima
MNNTIPRWRGFNLLEAFSIKSTGNFKEEDFLWMAQWDFNFVRIPMCHLLWSDRGNPFIIREDFFEKIDRVIFWGEKYGIHICISLHRAPGYSVNKEVEEKTNLWKDETAQEAFIHHWSFIARRYKGISSTHLSFNLINEPPFPDPQIMSVEDHNSLIKRTITEIRKIDPERLIIIDGLGYGNIPVDDLTIENTVQSCRGYIPFSVTHYKAEWVDSKDFPVPEWPNGWHFGEYWNREKLLEHYLTWIKLRQKGIEVFCGEMGAYNKTPHDVVLKWLEDLLEIFKTLNIGFALWNFRGPFGILDSERKDVEYEEWYGHKLDRKMLELLRKY

SEQ ID NO: 2 - protein, 239 aa, Aspergillus niger
MKLPVSLAMLAATAMGQTMCSQYDSASSPPYSVNQNLWGEYQGTGSQCVYVDKLSSSGASWHTEWTWSGGEGTVKSYSNSGVTFNKKLVSDVSSIPTSVEWKQDNTNVNADVAYDLFTAANVDHATSSGDYELMIWLARYGNIQPIGKQIATATVGGKSWEVWYGSTTQAGAEQRTYSFVSESPINSYSGDINAFFSYLTQNQGFPASSQYLINLQFGTEAFTGGPATFTVDNWTASVN

SEQ ID NO: 3 - protein, 343 aa, Bacillus amyloliquefaciens
MKHTMELIKKLVSIPSPTGNTYEVIAYTESLLKDWGVSSYRNRKGGLFVTIPGRDDKKHRLLTAHVDTLGAMVKEIKADGRLKIDLIGGFRYNSIEGEYCEIQTSSGKTYTGTILMHQTSVHVYKDAGKAERNQENMEVRLDEHVRTKEETSDLGIRVGDFISFDPRVQITPSGFIKSRHLDDKASVALLLDLIRRITEEKIDLPYTTHFLISNNEEIGYGGNSNIPPETVEYLAVDMGAIGDGQSTDEYTVSICVKDASGPYHYQLRKHLAGLAERYGIDYQLDIYPYYGSDASAAIRSGHDIVHGLIGPGIDASHAFERTHELSLLNTAKLLHRYVLSPMA